(19) United States
(12) Patent Application Publication
(10) Pub. No.: US 2021/0077540 A1
(43) Pub. Date: Mar. 18, 2021

(54) BIOACTIVE GREEN-LIPPED MUSSEL EXTRACTS AND USES THEREOF

(71) Applicant: SANFORD LIMITED, Freemans Bay Auckland (NZ)

(72) Inventor: Hong (Sabrina) TIAN, Freemans Bay Auckland (NZ)

(73) Assignee: SANFORD LIMITED, Freemans Bay Auckland (NZ)

(21) Appl. No.: 17/042,445

(22) PCT Filed: Mar. 27, 2019
(86) PCT No.: PCT/NZ2019/050033

§ 371 Date: Sep. 28, 2020

(30) Foreign Application Priority Data
Mar. 27, 2018 (NZ) 741103

(51) Int. Cl. A61K 35/618; A61K 38/05; A61K 38/06; A61K 38/07; A61K 38/08; A61P 39/06; A61P 9/12

(52) U.S. Cl. CPC A61K 35/618; A61K 38/05; A61K 38/06; A61P 9/12; A61K 38/08; A61P 39/06; A61K 38/07

(57) ABSTRACT

A biologically active non-lipid extract comprising of an isolated molecular weight fraction of <10 kDa or <1 kDa derived from New Zealand green-lipped mussels (Perna canaliculus). The extract exhibits biological activity selected from one or more of antihypertensive activity, antioxidant activity, antimicrobial activity, antiviral activity, and antiparasitic activity. The extract includes a plurality of biologically active substances selected from the group having free amino acids; peptides; cryptides; sugars and/or sugar-containing compounds including nucleosides and their derivatives; carbohydrates including glycoconjugates such as glycosides, glycosylamines, glycoproteins, glycopeptides, peptidoglycans; nitrogen-containing compounds including purines; phenolic compounds; minerals; metabolites.

### FIELD OF INVENTION

The present invention relates to biologically active mussel extracts obtained from the New Zealand green-lipped mussel species (Perna canaliculus) and uses thereof.

### BACKGROUND OF INVENTION

Extensive research has been carried out over the years in relation to the potential health benefits and bioactive properties of extracts of shellfish and other marine species (Sularia et. al., (2015) Marine-Based Nutraceuticals: An Innovative Trend in the Food and Supplement Industries Mar. Drugs (2015) 13, 6336-6351. The unique properties of the New Zealand green-lipped mussel (Perna canaliculus) have been studied for more than 40 years. It was observed that New Zealand coastal Maori populations historically had lower incidences of arthritis than inland Maori populations. This was attributed to the high consumption of green-lipped mussels by the coastal Maori populations thereby suggesting that the green-lipped mussel species had anti-inflammatory activity. Various dried green-lipped mussel powders (whole powders including lipid components) have been sold as health supplements for the treatment of arthritis in humans and animals.

Some clinical trials have since shown that lipid extracts of Perna canaliculus have anti-inflammatory activity and can be used in the management of arthritis (Halpern (2000) Anti-inflammatory effects of a stabilized lipid extract of Perna canaliculus (Lyprinol); Brien et al. (2008) Systematic review of the nutritional supplement Perna Canaliculus (green-lipped mussel) in the treatment of osteoarthritis Q J Med 2008; 101:167-179). Lipid extracts of green-lipped mussels have been commercialised for use in the relief of arthritic symptoms.

The New Zealand green-lipped mussel (Perna canaliculus) contains high levels of Omega-3 fatty acids and they are a rich source of other beneficial compounds including vitamins, minerals, taurine, amino acids, polyphenols, carotenoids and active compounds of glucosaminoglycan (GAG or mucopolysaccharide), collagen and glycogen, some of which have been shown to have positive health effects (Grienke et al. (2014) Bioactive compounds from marine mussels and their effects on human health Food Chemistry 142 (2014) 48-60; Coulson et al in Rainsford et al (2015) Novel Natural Products: Therapeutic Effects in Pain, Arthritis and Gastro-intestinal Diseases, Progress in Drug Research 70).

However, research in respect of green-lipped mussel powder supplements has predominantly been focused on the anti-inflammatory properties of lipid mussel extracts (Coulson et al (2015)), because it is widely believed that the anti-inflammatory properties are attributed to the lipid fraction. There has been little emphasis or research into the potential health properties of the non-lipid components present in mussel powders or extracts, in particular, the aqueous or hydrophilic fraction including water-soluble substances such as proteins, peptides and other potentially bioactive substances, and non-soluble substances such as high molecular weight components and un-dissolved proteins. It is widely believed that it is the lipid components present in the mussels that are the key components responsible for any biological activity.

For this reason, during mussel processing, the non-lipid components are generally discarded or used to make low value by-products such as defatted mussel powders that are not fit for pharmaceutical or nutraceutical use, but are instead used as food flavourings or seasonings, in animal feeds, in fishing bait and in fertilisers. Current commercially available defatted mussel powders have no known or proven bioactivity because they are by-products that are not produced for this purpose, and bioactive substances are not maintained or preserved in the products.

Given there are much higher volumes of non-lipid components in mussels in comparison to lipid components, discarding the non-lipid by-products or using them in low value end products is wasteful and a loss of opportunity to harness the potential health benefits of the entire mussel. Consequently, there is a need to investigate potential bioactivities and uses of non-lipid green-lipped mussel components in order to reduce waste and provide more high value and useful end products.

### OBJECT OF THE INVENTION

It is an object of the invention to provide biologically active extracts derived from non-lipid components of New Zealand green-lipped mussels, and/or compositions comprising said extracts, or at least to provide the public with a useful choice.

### SUMMARY OF INVENTION

In a first aspect the invention resides in a biologically active non-lipid extract consisting of an isolated molecular weight fraction of <10 kDa or <1 kDa derived from New Zealand green-lipped mussels (Perna canaliculus) wherein the extract exhibits biological activity selected from one or more of the group comprising antioxidant activity, antihypertensive activity, antimicrobial activity, antiviral activity, and antiparasitic activity.

Preferably the <10 kDa extract comprises a plurality of biologically active substances selected from the group comprising free amino acids; peptides; cryptides; sugars and/or sugar-containing compounds including nucleosides and their derivatives; carbohydrates including glycoconjugates such as glycosides, glycosylamines, glycoproteins, glycopeptides, peptidoglycans; nitrogen-containing compounds including purines; phenolic compounds; minerals; metabolites.

Preferably the <1 kDa extract comprises a plurality of biologically active substances selected from the group comprising free amino acids; small peptides such as dipeptides, tripeptides, tetrapeptides, pentapeptides; small cryptides; small sugars and/or sugar-containing compounds including nucleosides and their derivatives; small nitrogen-containing compounds including purines; small phenolic compounds; minerals; small molecule metabolites.

Preferably the extract comprises a plurality of free form amino acids including essential amino acids.

Preferably the extract comprises between about 1-10% by weight of free form amino acids.

More preferably the extract comprises between about 4-9% by weight of free form amino acids.

Preferably the extract comprises a higher proportion of Arginine and/or Glycine relative to the other amino acids.

In a further aspect the invention resides in a composition or preparation comprising the biologically active extract described herein. The composition or preparation is preferably a food product, a pharmaceutical, a medicament, a nutraceutical, a dietary supplement, a veterinary product, a cosmeceutical or a cosmetic preparation.

In a further aspect the invention resides in an antihypertensive composition comprising a biologically active extract as described herein. Preferably the antihypertensive activity is provided by one or more free form amino acids and/or one or more peptides and/or one or more cryptides present in the extract. Preferably the antihypertensive activity is provided by an ACE inhibitory effect.

Preferably the extract comprises at least 30% potentially bioactive antihypertensive peptides.

Preferably the extract comprises a plurality of peptides, at least one of which is selected from the group comprising peptides having amino acid sequences: Phe-Phe; Leu-Asp-Leu; Leu-Glu-Leu; Leu-Gly-Leu; Leu-Asn-Phe; Leu-Thr-Phe; Leu-Trp; Val-Asp-Phe; Val-Asp-Trp; Val-Glu-Phe; Leu-Leu-Phe; Leu-Trp-Phe. More preferably, the extract comprises at least one peptide selected from the group comprising Leu-Leu-Phe; Leu-Asn-Phe; Leu-Thr-Phe; and Leu-Trp.

In a further aspect the invention resides in an ACE inhibitory peptide isolated from New Zealand green-lipped mussels (Perna canaliculus), wherein the peptide comprises an amino acid sequence selected from the group consisting of Leu-Leu-Phe; Leu-Asn-Phe; Leu-Thr-Phe; and Leu-Trp. The isolated peptide(s) can be incorporated into compositions, including functional foods and beverages for treating, regulating or preventing high blood pressure and/or hypertension.

In a further aspect the invention resides in the use of a biologically active extract as described herein in the manufacture of a composition or medicament for the treatment, regulation or prevention of high blood pressure or hypertension.

In still a further aspect the invention resides in a method of treating, regulating or preventing high blood pressure or hypertension by administering to a subject in need thereof, a therapeutically effective amount of a biologically active extract or composition as described herein.

In a further aspect the invention resides in an antioxidant composition comprising a biologically active extract as described herein. Preferably the antioxidant activity is provided by one or more free form amino acids, and/or one or more peptides, and/or one or more cryptides, and/or one or more sugars or sugar-containing compounds such as nucleosides or their derivatives, and/or one or more nitrogen-containing compounds such as purine derivatives, present in the extract.

Preferably the extract comprises at least 5% potentially bioactive antioxidative peptides.

Preferably the antioxidant composition comprises a plurality of peptides, including at least one peptide having an amino acid sequence selected from: Leu-Val-Ser-Lys and/or Leu-Tyr-Glu-Gly-Tyr.

Preferably the antioxidant composition comprises an isolated extract with molecular weight <1 kDa. Preferably the extract exhibits DPPH scavenging activity.

The extract or antioxidant composition may be used as a natural antioxidant in the preservation of a wide range of products including food products, cosmetics, and pharmaceutical products.

In a further aspect the invention resides in an antimicrobial composition comprising a biologically active extract as described herein. The extract or antimicrobial composition may be used as a natural antimicrobial in the preservation of a wide range of products including food products, cleaning products, cosmetics, and pharmaceutical products.

In a further aspect the invention resides in an antiviral composition comprising a biologically active extract as described herein. An extract having antiviral properties can be included in a variety of health products.

In a further aspect the invention resides in an antiparasitic composition comprising a biologically active extract as described herein. An extract having antiparasitic properties can be included in a variety of health products and veterinary products.

In a further aspect the invention resides in a pharmaceutical composition which comprises a biologically active extract as described herein and one or more pharmaceutically acceptable excipients.

In a further aspect the invention resides in a nutraceutical composition which comprises a biologically active extract as described herein and one or more nutraceutically acceptable excipients. The nutraceutical composition may be a dietary or nutritional supplement.

In a further aspect the invention resides in a food composition which comprises a biologically active extract as described herein. The food composition could be a functional food or beverage, a functional food or beverage ingredient, a functional food or beverage additive, or a dietary supplement.

In still a further aspect the invention resides in the use of a biologically active extract as described herein, in the manufacture of an antioxidant composition.

In still a further aspect the invention resides in the use of a biologically active extract as described herein in the manufacture of an antimicrobial composition.

In still a further aspect the invention resides in the use of a biologically active extract as described herein in the manufacture of a pharmaceutical composition, veterinary composition, nutraceutical composition, cosmeceutical composition, food product or cosmetic product.

### Definitions

In this specification, unless the context otherwise requires, the following terms shall have the following definitions:

“crude extract” means a whole (unfractionated) extract derived from green-lipped mussels (including or excluding the shells) in any form, or a defatted or delipidated extract derived from green-lipped mussels in any form.
“hydrophilic fraction” means the fraction of an extract remaining after at least one lipid removal, separation or extraction step has been carried out on a crude whole mussel extract, said fraction including predominantly non-lipid components and non-lipid molecules, however some hydrophobic substances may still remain in the fraction.
“biologically active extract” means an extract that exerts a pharmacological (or biochemical and/or physiological) effect on a gene expression, cell, tissue, organ or organism.
“cryptides” means potential bioactive peptides hidden or encrypted within the sequence of a parent protein.

### DESCRIPTION

The invention will now be described, by way of example only, with reference to the accompanying drawings:

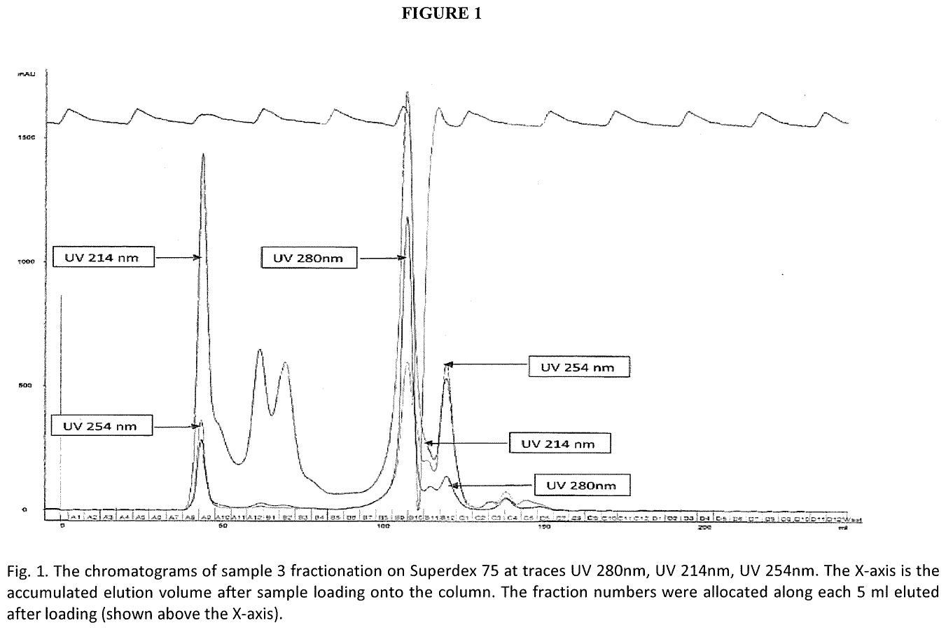

FIG. 1 shows a chromatogram of Superdex 75 fractionation of test sample 3 as referenced in Example 3;

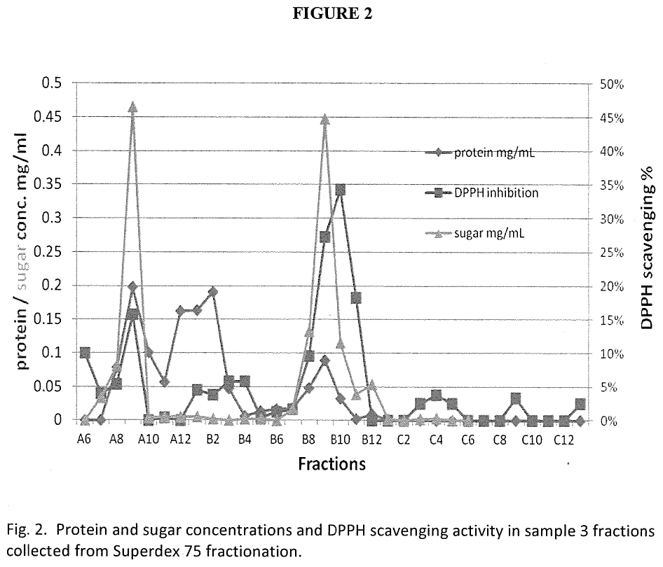

FIG. 2 is a graph showing protein and sugar concentrations and DPPH scavenging activity in test sample 3 fractions collected from Superdex 75 fractionation;

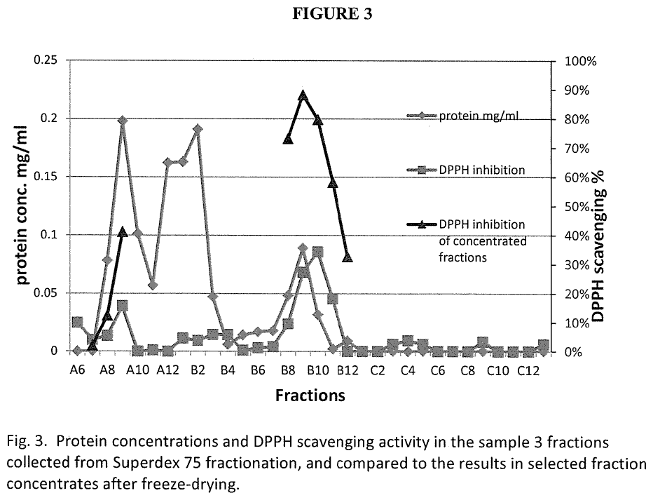

FIG. 3 is a graph showing protein concentrations and DPPH scavenging activity in the sample 3 fractions collected from Superdex 75 fractionation, and compared to the results in selected fraction concentrates;

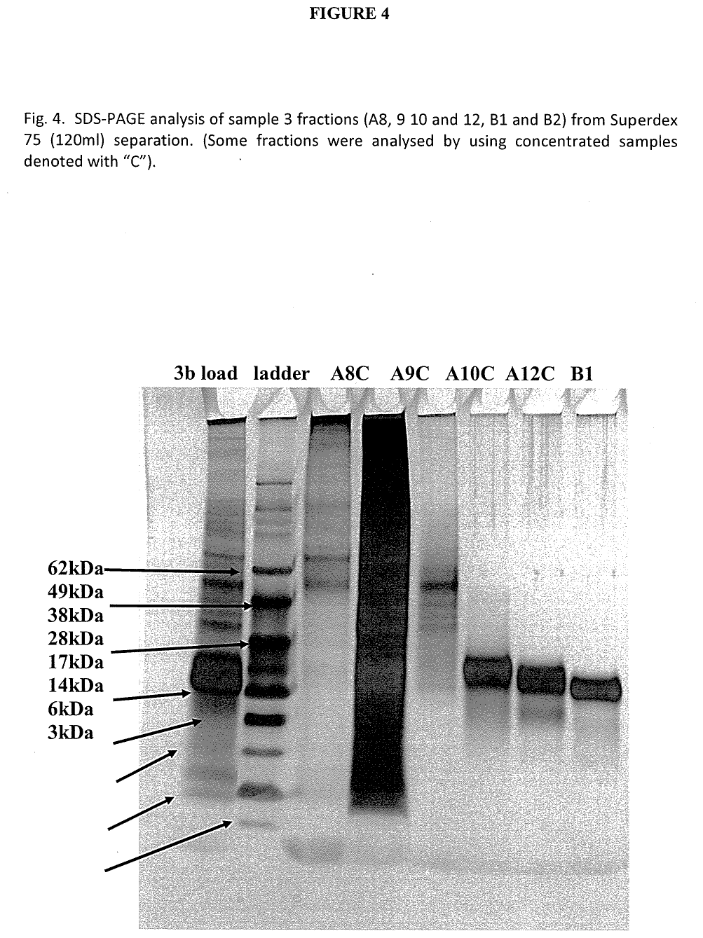

FIG. 4 is an SDS-PAGE analysis of sample 3 fractions (A8, 9 10 and 12, B1 and B2) from Superdex 75 fractionation;

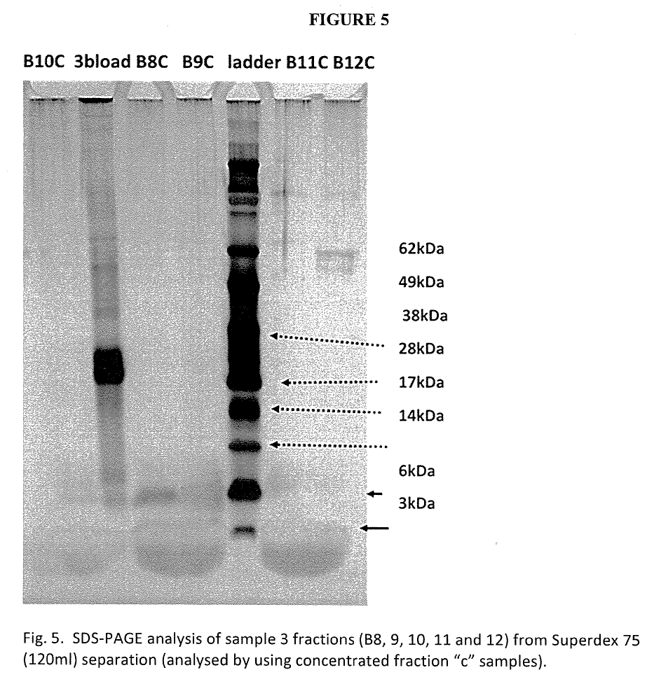

FIG. 5 is an SDS-PAGE analysis of sample 3 fractions (B8, 9, 10, 11 and 12) from Superdex 75 fractionation;

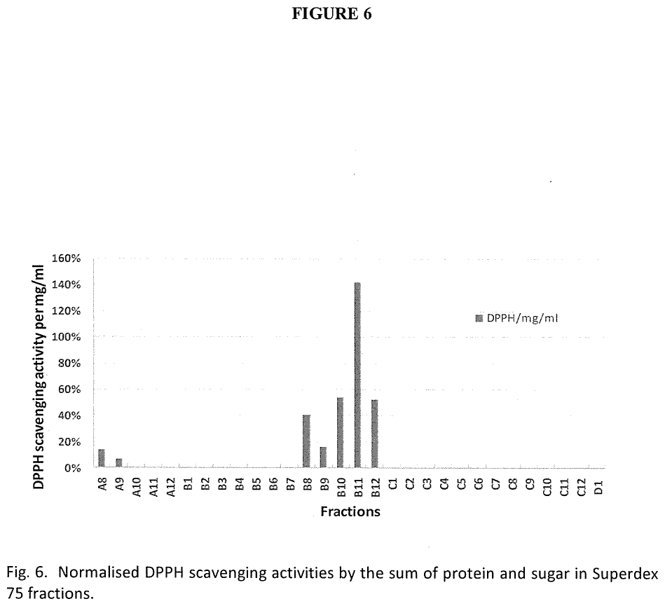

FIG. 6 is a graph showing the normalised DPPH scavenging activities by the sum of protein and sugar concentrations;

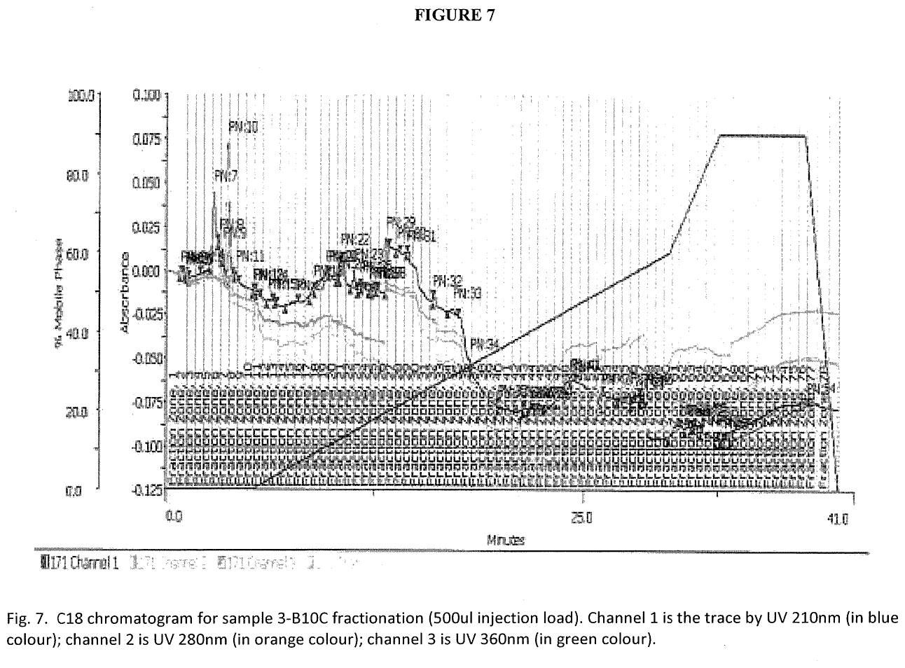

FIG. 7 is a C18 chromatogram for sample 3-B10C fractionation;

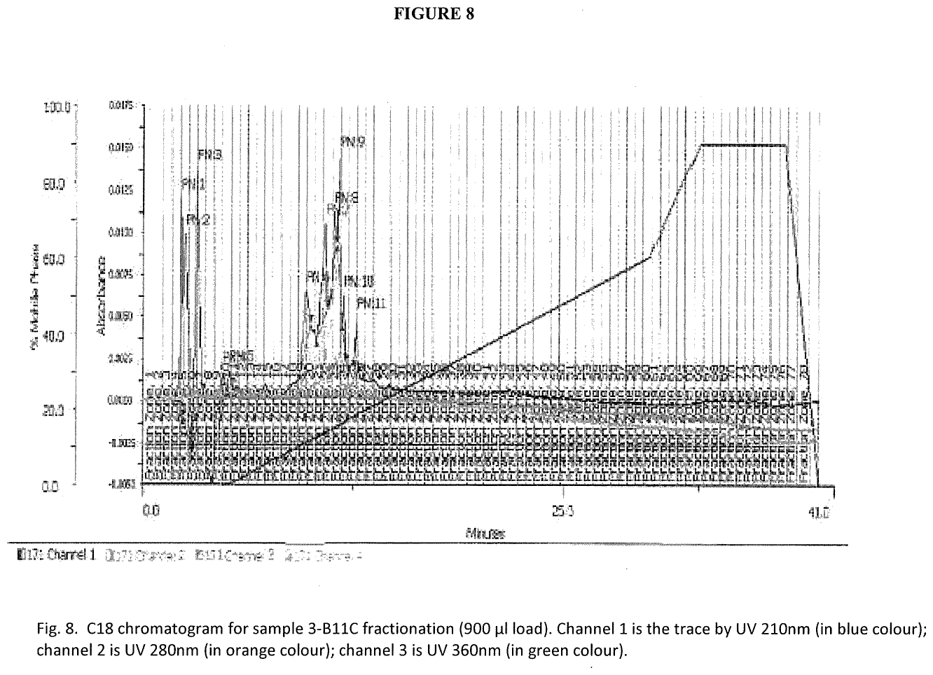

FIG. 8 is a C18 chromatogram for sample 3-B11C fractionation;

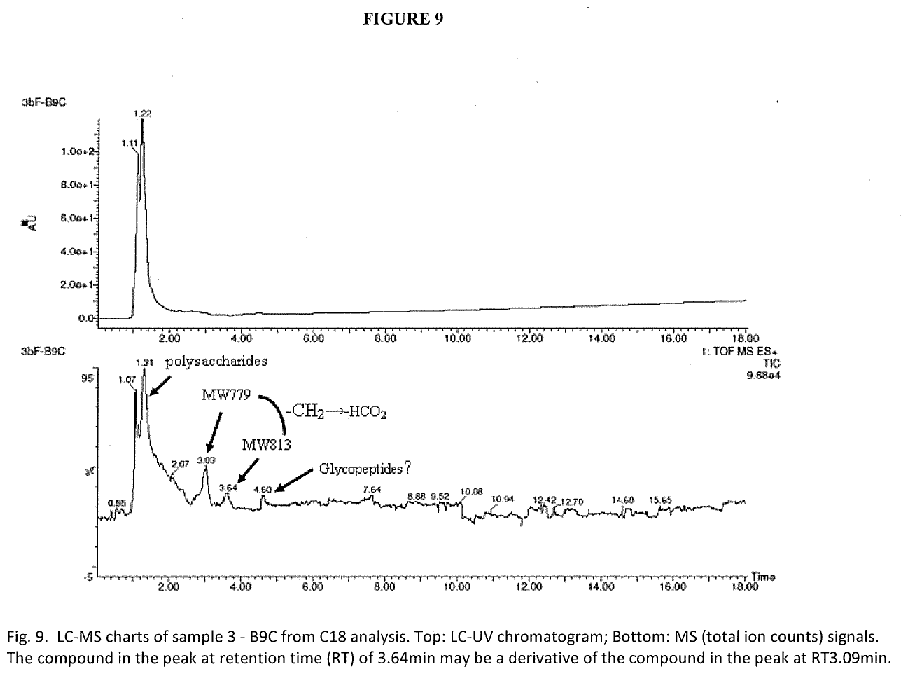

FIGS. 9-12 show the LC-MS results for the Superdex 75 fractions, B9C to B12C;

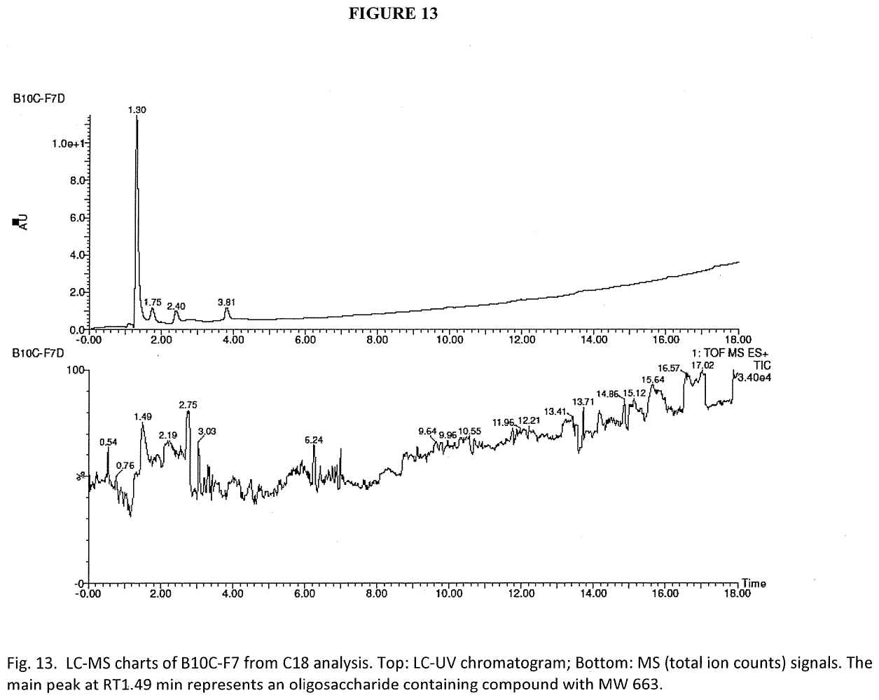

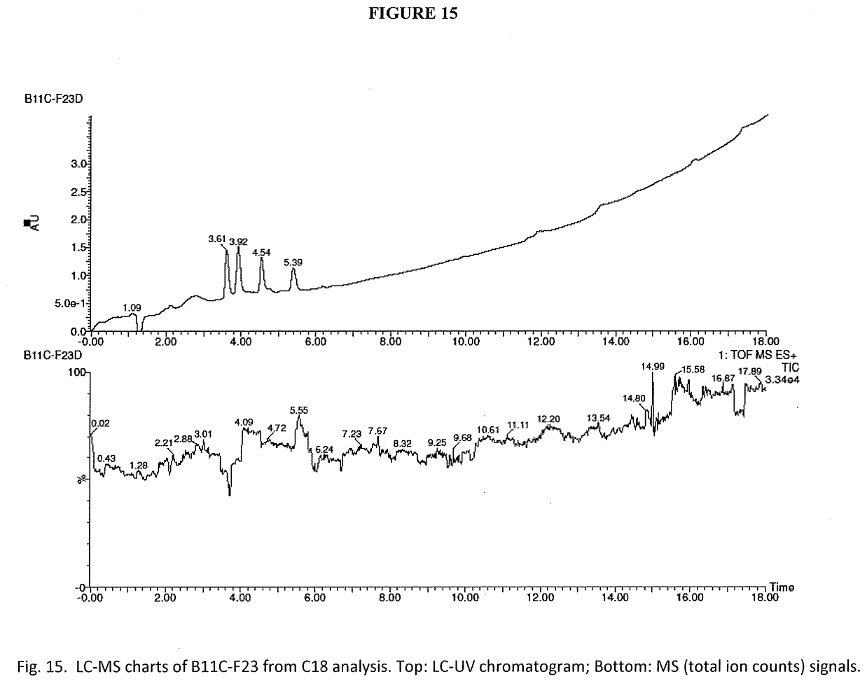

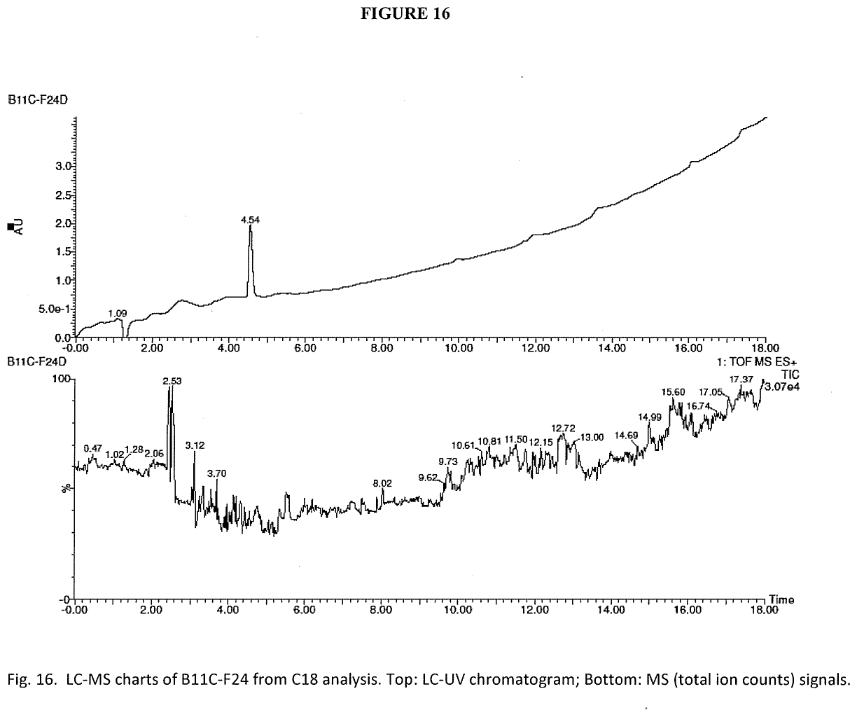

FIGS. 13-16 show the LC-MS results from C18 analysis of B10C-F7 and B11C-22, 23 and 24;

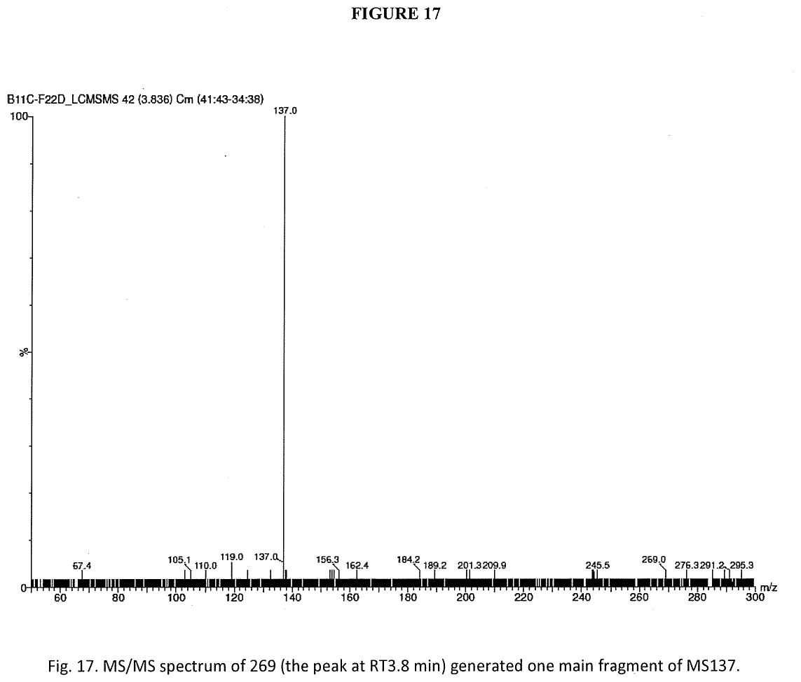

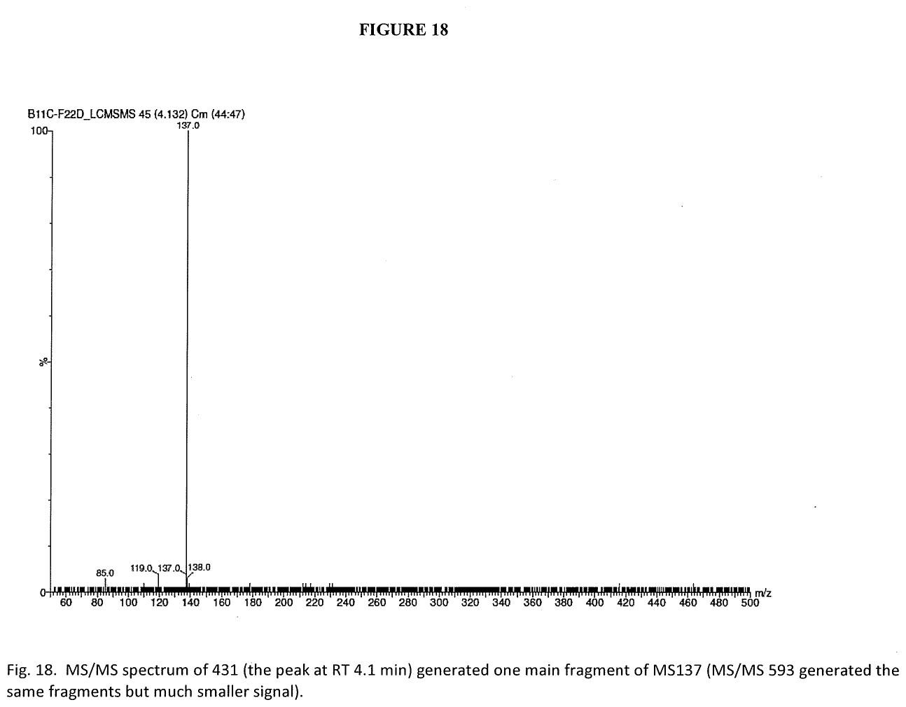

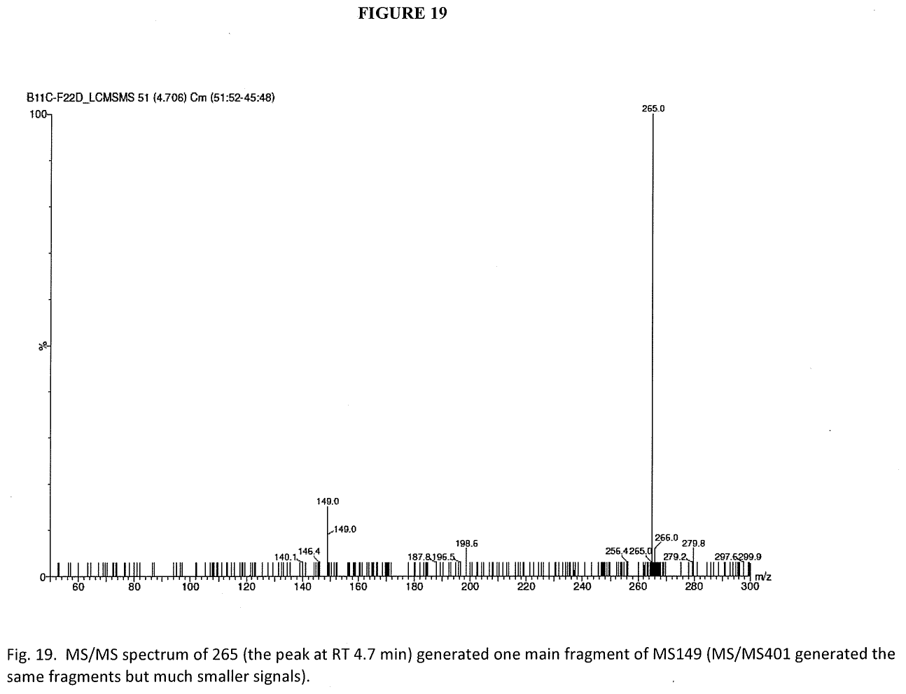

FIGS. 17-22 show the MS/MS analysis of certain biologically active compounds;

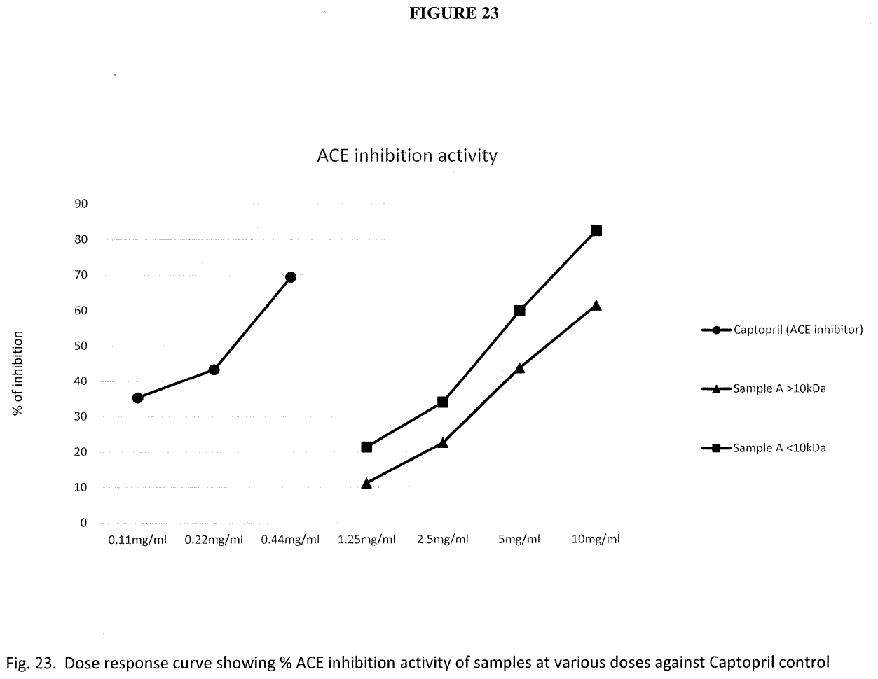

FIG. 23 is a graph showing the ACE inhibition dose response curve of an extract of the invention;

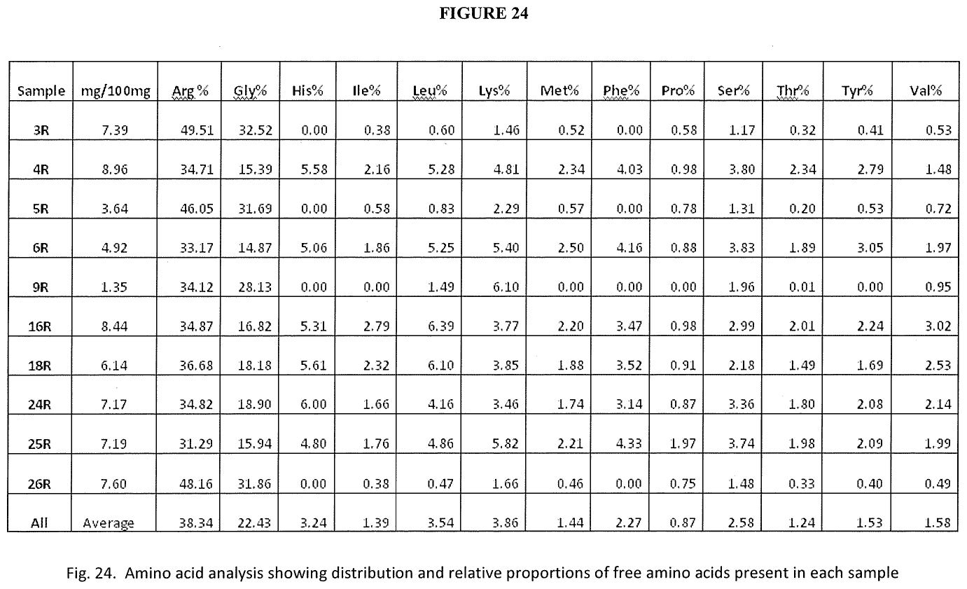

FIG. 24 is a table showing the distribution and relative proportions of free form amino acids present in the extracts tested;

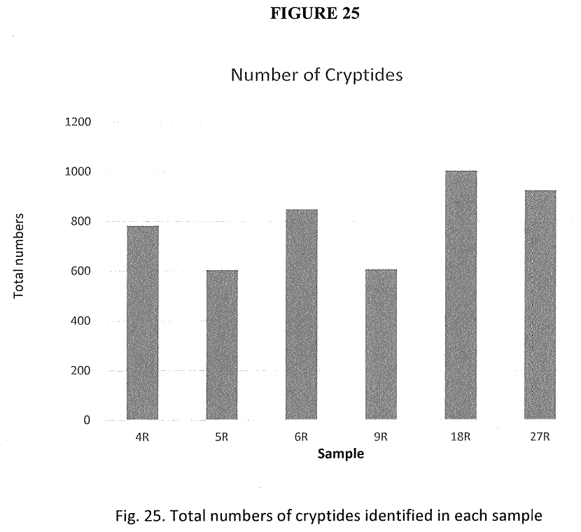

FIG. 25 is a graph showing the total numbers of potential bioactive peptides present in the extracts tested;

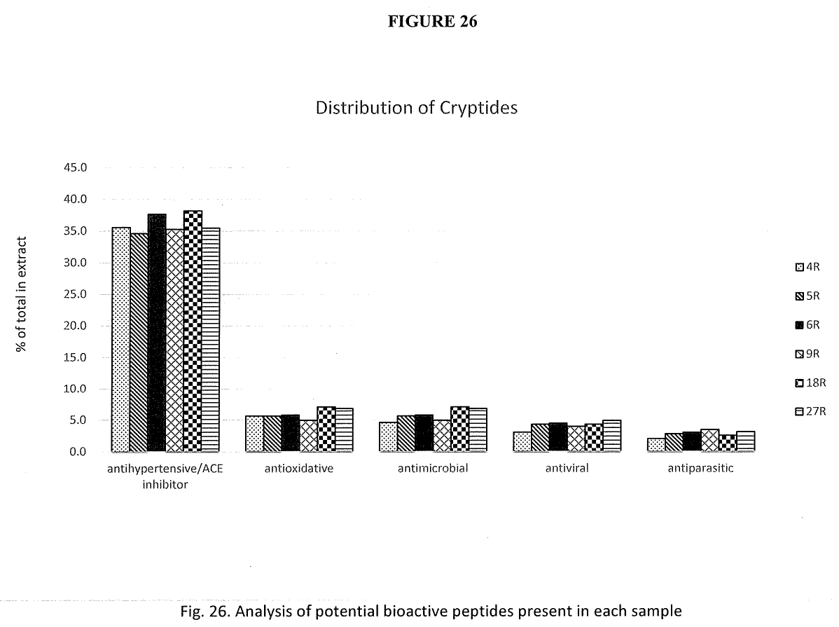

FIG. 26 is a graph showing the type and relative proportions of potential bioactive peptides present in the extracts tested.

The following description will describe the invention in relation to preferred embodiments of the invention, however the invention is in no way limited to these preferred embodiments as they are purely to exemplify the invention and it is envisaged that possible variations and modifications could be made that would be readily apparent to those skilled in the art without departing from the scope of the invention.

The invention relates to a biologically active extract derived from New Zealand green-lipped mussel (Perna canaliculus), wherein the extract comprises substantially non-lipid or hydrophilic components obtained from a crude mussel extract. It has been surprisingly found that the non-lipid components extracted from green-lipped mussels, exhibit certain bioactivity, including antihypertensive, antioxidant, antimicrobial, antiviral and antiparasitic activity.

The extract of the invention can be obtained from any green-lipped mussel starting material, including live mussels, fresh mussels, frozen mussels, with or without shells, to produce a crude whole (unfractionated) green-lipped mussel extract or composition. Alternatively, the extract of the invention can be obtained from a liquid, semi-dried or dried crude whole (unfractionated) mussel composition, or from any liquid, semi-dried or dried green-lipped mussel extract or composition that has already had the lipid components substantially removed (e.g. defatted or delipidated mussel extracts, compositions or powders). It is important that the crude mussel composition used to obtain the extract of the invention has been processed in a manner which has conserved the majority of bioactive components present in the mussel starting material. For example, it is important that excessive heat is not used during processing as that will destroy the bioactive components present in the mussel starting material. Preferably gentle low temperature processing methods are used to produce the crude extract in order to conserve as many of the bioactive components as possible.

Processing methods for producing whole mussel compositions or extracts typically involve the following steps: (1) removal of the flesh or meat from the shells of the mussels—for example, this can be done manually, or mechanically using for example, crushing or mechanical opening or shucking techniques, or by a high pressure process, followed by separation of the shells; (2) size reduction to reduce the flesh or meat into small particles—this can be done by homogenising techniques including mechanical homogenisation such as mincing, grinding, blending, centrifuging or pulverising the flesh or meat of the mussels, or alternatively, the flesh or meat could be liquefied by other means, including biotransformation processes including enzyme hydrolysis, acid or alkali hydrolysis, or fermentation.

In the applicant's patent application number PCT/NZ2017/050167 an enzymatic processing method is described which can be carried out on whole, live mussels, and which comprises opening or gapping the mussels (preferably by gentle warming) and exposing one or more target substrates of the live mussels to an enzyme formulation for a sufficient period of time to liquefy the target substrate(s) into the form of an emulsion, followed by removal of residual shells, shell fragments, and non-target substrates or non-target biological material. This is a preferred method of producing a crude whole mussel extract because it produces both high yields of extract (quantity) and high yields of biologically active material (quality).

For example, the applicant's research shows that mussel extracts produced by the enzyme hydrolysis process described in PCT/NZ2017/050167 generally have higher levels of bioactivity in comparison to crude mussel extracts produced by other processes.

Typically, crude mussel compositions (either whole compositions or “defatted” compositions) are dried and then ground or milled into a powder format. Generally low temperature drying methods such as freeze drying or spray drying are used, but other drying methods such as flash drying, vacuum drying or belt drying may be used.

The biologically active extract of the invention is made by carrying out at least one separation, fractionation or extraction step on a crude mussel composition or extract in order to isolate the desired biologically active fraction therefrom.

If the crude extract is a whole mussel extract or composition, at least one separation step may be carried out initially to separate the whole mussel composition or extract into its main fractions, that is, the lipid-rich or hydrophobic fraction; and the non-lipid or hydrophilic fraction (which may still contain some hydrophobic substances). This can be achieved by methods known in the art, such as aqueous or solvent extraction methods; siphoning or pumping off the lipid fraction; centrifugation; decanting; tricanting; precipitation or crystallisation methods; solid phase extraction (SPE) methods; gel filtration or size exclusion chromatography (SEC) methods; ion-exchange chromatography; ultrafiltration; or nanofiltration.

After the separation step, one or more fractionation steps is carried out to isolate specific molecular weight fractions from the crude extract, namely <10 kDa and <1 kDa fractions, using methods known in the art.

Alternatively, specific molecular weight fractions, namely <10 kDa and <1 kDa fractions, can be isolated from the crude extract without first separating the lipids and non-lipids, as the lipid components having higher molecular weights will naturally remain in the retentate. The fractionation steps can be carried out by methods known in the art, including solid phase extraction methods, membrane filtration methods, ultrafiltration methods, nanofiltration methods, chromatography methods including liquid chromatography, gas chromatography, affinity chromatography, SEC (gel filtration), HPLC, and ion-exchange chromatography.

The isolated fraction can be concentrated by methods known in the art (for example, low-temperature vacuum evaporation) to produce a concentrated biologically active extract of the invention. The isolated fraction can also be readily purified if desired by employing additional purification steps according to methods known in the art.

The biologically active extract of the invention is preferably dried, for example, using a low temperature drying method such as freeze drying, or other flash drying techniques such as spray drying, vacuum drying or belt drying. This enables the extract to be readily used and incorporated into various products forms.

An example of one preferred method of making the extract of the invention is described as follows. A crude whole mussel extract is prepared using the enzymatic processing method described in PCT/NZ2017/050167. That is, whole, live mussels, are opened or “gapped” and exposed to an enzyme formulation for a sufficient period of time, and at a sufficient temperature, to liquefy the mussels into the form of an emulsion, followed by separation of any residual shells and/or shell fragments and other non-target biological material. Preferably the enzyme formulation comprises at least one protease derived from Bacillus amyloliquefaciens. Preferably the mussels are exposed to the enzyme(s) for at least 50 minutes at a temperature of between 50-55° C. The liquid mussel composition produced by this process can then be used to prepare the extract of the invention by carrying out at least one separation, fractionation or extraction step using ultrafiltration methods on the liquid composition to recover the desired fractions comprising compounds having molecular weights of <10 kDa or of <1 kDa. Alternatively, the liquid composition can be dried, for example, by freeze drying or spray drying, and optionally milled or ground into a powder, before ultrafiltration is carried out to recover the desired fraction.

The biologically active extract of the invention comprises substantially non-lipid components such as proteins, cryptides, peptides, free amino acids, nucleic acids, minerals, sugars or sugar containing compounds such as nucleosides and their derivatives, carbohydrates including glycoconjugates such as glycosides, glycosylamines, glycoproteins, glycopeptides, peptidoglycans, nitrogen-containing compounds including purine derivatives, phenolic compounds and other small molecule metabolites. Some small hydrophobic substances may still be present in the extract.

It has been surprisingly found that the extract of the invention comprises a plurality of biologically active components having a number of potential uses, for example, as an antihypertensive agent, an antioxidant, an antimicrobial agent, an antiviral agent, and/or an antiparasitic agent.

The terms “anti-hypertensive agent” and “anti-hypertensive composition” as used herein, relate to substances or compositions that can be used for the treatment or prevention of high blood pressure, or hypertensive conditions generally, including the treatment or prevention of conditions that may be caused by or result from high blood pressure or hypertension generally.

The terms “antioxidant” and “antioxidant composition” as used herein, relate to a substance or composition that is able to reduce oxidative stress by fully or partially inhibiting oxidation and/or oxidation processes such as inhibition of enzymes, which are involved in the oxidation pathways, or that can remove potentially damaging oxidizing agents such as free radicals in a living organism.

The terms “antimicrobial agent” and “antimicrobial composition” as used herein, relate to a substance or composition that is capable of fully or partially destroying or inhibiting the growth of microorganisms, particularly pathogenic microorganisms.

The terms “antiviral agent” and “antiviral composition” as used herein, relates to a substance or a composition that assists in countering viral effects or infection.

The terms “antiparasitic agent” and “antiparasitic composition” as used herein, relates to a substance or composition that assists in countering parasitic infection.

It is envisaged that the biologically active extracts of the invention could be formulated into a wide variety of compositions for these uses. For example, the extracts could be used in food applications as functional food or beverage formulations, food or beverage ingredients, functional food flavourings or seasonings, or they could be used in the manufacture of cosmetics (e.g. as antioxidants), or in the manufacture of pharmaceutical, nutraceutical or dietary supplement compositions such as tablets, capsules, cachets, syrups, elixirs, or other dosage forms using suitable carriers and excipients as required. The extracts could also be used in veterinary applications, such as nutritional pet foods, dietary supplements and veterinary medicines.

For example, a non-lipid isolated active fraction derived from green-lipped mussels could be used in various antioxidant applications including:

food applications (as additives or preservatives to ensure that foodstuffs keep their taste and colour and remain edible over a longer period, and to prevent oxidation occurring which can destroy certain vitamins and amino acids present in the foodstuff);
pharmaceutical and/or nutraceutical applications including dietary or nutritional supplements or functional foods or beverages (antioxidants have been found to be very important to good health because the counteract the damaging effects of free radicals which can cause a wide range of illnesses and chronic diseases);
cosmetic applications (antioxidants have been shown to help protect the skin from sun damage and premature aging).

As a further example, an extract of the invention could be used in various antimicrobial applications including food applications, pharmaceutical applications, cleaning applications, personal care applications and industrial applications.

As a further example, an extract of the invention could be used in various applications for antihypertensive purposes including:

functional food applications (as functional ingredients in food or beverage products specially designed to treat, regulate or prevent high blood pressure or hypertension);
pharmaceutical and/or nutraceutical applications including dietary or nutritional supplements (designed to treat, regulate or prevent high blood pressure or hypertension).

Similarly, an extract of the invention could be used in various applications for antiviral or antiparasitic purposes.

Accordingly, the invention provides for compositions containing the biologically active extract of the invention, optionally with conventional additives and/or excipients, including physiologically acceptable carriers, preservatives, buffers, stabilisers and the like as required depending on the dosage form.

The following examples are provided for illustrative purposes only.

### Example 1—Anti-Hypertensive Activity

There are significant and increasing numbers of hypertensive adults worldwide, making hypertension prevention, treatment and control a high priority in public health systems, particularly since high blood pressure increases cardiovascular risk and risk of other health issues. The control of hypertension is generally associated with the renin angiotensin aldosterone system (RAAS) as well as the nitric oxide (NO) system and the sympathetic nervous system (SNS) system. Key enzymes within the RAAS include renin, which acts on angiotensinogen produced by the liver to yield angiotensin-I, and angiotensin-I-converting enzyme (ACE). ACE is a dipeptidyl carboxypeptidase which releases C-terminal His-Leu from decapeptide angiotensin I and converts it into angiotensin II. Angiotensin II is a powerful vasoconstrictor and salt-retaining peptide. Abnormally high levels of angiotensin II will cause high blood pressure, and lead to diseases, such as pulmonary arterioles and sarcoidosis.

Currently, several synthetic peptides are used in the clinical treatment of high blood pressure. These include the sulfhydryl-containing agents including Captopril, the first ACE inhibitor, Zofenopril, dicarboxylate-containing agents including Enalapril, Ramipril, Quinapril, Perindopril, Lisinopril, and Benazepril, and phosphonate-containing agents including Fosinopril. However, synthetic ACE inhibitors have several undesirable but common side effects including coughing, dizziness, headaches, rashes, chest pain and adverse interactions with other medications. In addition, they cannot be used in pregnant women as they may cause birth defects. Therefore, research has been focused on finding safer alternatives to these synthetic drugs, including investigating food therapy and dietary approaches to preventing hypertension.

In order to investigate potential antihypertensive effects of New Zealand green-lipped mussel extracts, the following non-lipid extracts obtained from dried crude whole mussel compositions were tested for antihypertensive activity, by determining whether they showed any inhibition activity against ACE.

| Sample No. | Description |
|---|---|
| 1 | Dried composition produced by mechanical opening |
| | of mussels, removal of meat, homogenising of meat |
| | and spray drying. The lipid rich fraction was removed |
| | by one EtOH solvent extraction step and the remaining |
| | hydrophilic fraction was recovered. |
| 2 | Dried composition produced by mechanical opening |
| | of mussels, removal of meat and freeze drying. |
| | The lipid rich fraction was removed by one EtOH solvent |
| | extraction step and the remaining hydrophilic |
| | fraction was recovered. |
| 3 | Dried composition produced by mechanical opening |
| | of mussels, removal of meat and homogenisation |
| | by mincing followed by freeze drying. The lipid rich |
| | fraction was removed by one EtOH solvent extraction |
| | step and the remaining hydrophilic fraction |
| | was recovered. |
| 4 | Dried composition produced using enzymatic process |
| | described in PCT/NZ2017/050167 to produce a liquid |
| | composition, followed by freeze drying and fractionation |
| | by one EtOH solvent extraction step and recovery of the |
| | remaining hydrophilic fraction. |
| 5 | Dried composition produced by using enzymatic process |
| | described in PCT/NZ2017/050167 to produce a liquid |
| | composition, followed by spray drying and fractionation |
| | by one EtOH solvent extraction step and recovery of the |
| | remaining hydrophilic fraction. |
| 6 | Dried composition produced by mechanical opening |
| | of mussels, removal of meat and homogenisation |
| | by mincing followed by fractionation by one DME |
| | solvent extraction step and recovery of the remaining |
| | hydrophilic fraction which was dried by freeze drying. |
| 7 | Dried composition produced by using enzymatic process |
| | described in PCT/NZ2017/050167 to produce a liquid |
| | composition, followed by freeze drying and fractionation |
| | by one DME solvent extraction step and recovery of the |
| | remaining hydrophilic fraction. |
| 8 | Dried composition produced using enzymatic process |
| | described in PCT/NZ2017/050167 to produce a liquid |
| | composition, followed by spray drying and fractionation |
| | by one DME solvent extraction step and recovery of |
| | the remaining hydrophilic fraction. |
| 9 | Liquid composition produced using enzymatic |
| | process described PCT/NZ2017/050167, followed by |
| | fractionation by one DME solvent extraction step, and |
| | recovery of the remaining hydrophilic fraction. |

In this study, an ACE assay was used to screen the inhibition activity of ACE in the above samples. The ACE substrate N-[3-(2-Furyl)acryloyl]-Phe-Gly-Gly (FAPGG) and ACE from rabbit lung were both purchased from Sigma. The working solution of FAPGG was prepared at 0.5 mM in TrisHCl buffer (pH8) containing 0.3M NaCl. The ACE was stocked in 2 U/ml solution and freshly diluted to 0.2 U/ml in the same TrisHCl buffer before the assay. Captopril, an ACE inhibitor, was prepared at 1 mM and used in the assay as the positive control.

The measurement of the ACE assay is based on the hydrolysis of FAPGG after addition of ACE. The released FAP will cause decline of absorbance at 340 nm. The assays were performed in a 96-well plate pre-warmed up to 37° C. An aliquot of 20 μl of the sample (in triplicates) was added into the well, followed by 20 μl of ACE and 180 μl of FAPGG. The absorbance at 340 nm was measured in a spectrophotometer (SpectraMax M4) at kinetic mode for 10 min at 37° C. The maximum hydrolysis rate (measured as the slope of the declined absorbance versus the 10 min time frame) was used as the ACE activity. The ACE inhibition was calculated as following:

$$ACE inhibition \% = ( 1 - \frac{Δsample}{Δcontrol} ) \times 100 ,$$

where Δsample is the slope of the hydrolysis in samples, Δcontrol is the speed of hydrolysis in control (no inhibitor).

All samples were diluted from 10 mg/ml soluble stock with assay buffer (50 mM Tris-HCl containing 0.3M NaCl at pH 8) to 5, 1, 0.1, 0.01 and 0.001 mg/ml. The initial tests at 10 mg/ml and 5 mg/ml revealed near 100% inhibition in samples 1 to 5, with further dilutions all samples started to show differences in ACE inhibition activity. The correlations between the ACE inhibition activity in each sample at different concentrations were plotted in Excel, and a math equation fit was performed in order to calculated the IC₅₀ value for each sample. The IC₅₀ values are listed in the table below:

TABLE 1
IC₅₀ values for ACE inhibition activity
of each hydrophilic extract sample

| Sample | 1 H | 2 H | 3 H | 4 H | 5 H | 6 H | 7 H | 8 H | 9 H |
|---|---|---|---|---|---|---|---|---|---|
| IC₅₀ | 3.3 | 9.6 | 4.9 | 10.5 | 10.2 | 5.6 | 5.3 | 6.2 | 5.8 |
| (μg/ml) | | | | | | | | | |

All samples demonstrated good ACE inhibition (>70%) at concentrations greater than 0.1 mg/ml.

As a comparison, the ethanol extract counterparts of each of the above samples (i.e. the lipid fraction separated from each sample by ethanol or DMSO extraction) were also tested using the same method and the results showed that the lipid fraction of each of the nine samples had much lower (500 fold) activities than their hydrophilic fraction counterparts. This is shown in the table below where the IC₅₀ value is represented in mg/ml as opposed to μg/ml in the above table.

TABLE 2
IC₅₀ values for ACE inhibition activity
of each corresponding lipid extract sample

| Sample | 1 L | 2 L | 3 L | 4 L | 5 L | 6 L | 7 L | 8 L | 9 L |
|---|---|---|---|---|---|---|---|---|---|
| IC₅₀ | 12.36 | 3.90 | 10.55 | 5.07 | 4.58 | 15.19 | 10.47 | 9.81 | 8.91 |
| (mg/ml) | | | | | | | | | |

In the ACE inhibition activity assay, the hydrophilic extract samples demonstrated IC₅₀ values at very low concentration (μg/ml level), in comparison to the lipid extract samples with IC₅₀ values at high concentrations (mg/ml level). This shows that the majority of the anti-hypertensive activity can be contributed to the hydrophilic components present in the green-lipped mussel compositions, not the lipid components.

### Example 2—ACE Inhibition Activity of Fractionated Samples

As a result of the studies described in Example 1, two fractionated green-lipped mussel extract samples were produced as follows:

| Sample No. | Description |
|---|---|
| A | Extract produced using enzymatic process described in |
| | PCT/NZ2017/050167 to produce a liquid whole |
| | mussel composition, followed by spray drying. Dried extract |
| | was rehydrated in water and fractionated by centrifugation |
| | to recover the hydrophilic fraction, which was then |
| | subjected to membrane filtration using 10 kDa |
| | ultracentrifugation. |
| B | Extract produced by mechanical opening of mussels, |
| | removal of meat, homogenising of meat and spray drying. |
| | Dried extract was rehydrated in water and fractionated by |
| | centrifugation to recover the hydrophilic fraction, |
| | which was then subjected to membrane filtration |
| | using 10 kDa ultracentrifugation. |

The 10 kDa filtration step was carried out using a small scale centrifugal filtration unit installed with a 10 kDa membrane (Amicon Ultra-0.5 mL, Millipore, USA). A total of 0.4 mL of each sample was loaded into the centrifugal unit which was then centrifuged for 10 min at 14,000×g. The retentate was approximately at 40 μL, ˜10 fold concentrated. Both filtrate and retentate were collected and tested for ACE inhibition activity using the same method as described in Example 1, except that a different buffer reagent (DMSO or a mix of DMSO with water) was used to solubilize the powder.

The fractions were diluted with water and tested at estimated 5 mg/mL level. The ACE inhibition activity results are summarised in the following table:

| | 10 kDa RTT (retentate after | 10 kDa FF (filtrate after |
|---|---|---|
| Sample | 10 kDa filtration) | 10 kDa filtration) |
| A | 34.08% | 67.77% |
| B | 48.57% | 57.03% |

The results showed that both samples had higher ACE inhibition activity in the isolated <10 kDa molecular weight fraction.

Based on these results, sample A was selected for further testing for dose response of ACE inhibition activity.

The two fractions: fraction >10 kDa and fraction <10 kDa were freeze-dried. The dry powders of the fraction <10 kDa were off white and slightly tinted in yellow. The dry powders of the fraction >10 kDa were light brown in colour. Both fractioned powders were resolubilised in water at 10 mg/mL first, then diluted to 5, 2 and 1 mg/mL in water for testing. Captopril (Sigma), a well-known angiotensin converting enzyme inhibitor, was used as a positive control and tested at 0.11, 0.22, 0.44 mg/mL. The results are summarised in the table below and shown in FIG. 23.

| Sample A | mg/mL | 10 | 5 | 2 | 1 | IC50 |
|---|---|---|---|---|---|---|
| | >10 kDa | 61.35% | 43.67% | 22.51% | 11.14% | 6.35 |
| | <10 kDa | 82.40% | 59.85% | 33.93% | 21.31% | 3.24 |
| Captopril | mg/mL | 0.44 | 0.22 | 0.11 | | 0.26 |
| (ACE | | 69.30% | 43.26% | 35.21% | | |
| inhibitor) | | | | | | |

### Example 3—Antihypertensive Compounds

In order to further elucidate the substances likely to be responsible for the antihypertensive and/or ACE inhibition activities of the hydrophilic fractions as shown in example 1, a peptide analysis was carried out on an isolated fraction of a hydrophilic mussel extract derived from New Zealand green-lipped mussels. The extract used in this analysis was produced from a whole green-lipped mussel composition which was produced by enzyme hydrolysis of the mussel meat/tissue using an enzyme derived from Bacillus amyloliquefaciens (commercially available as NEUTRASE). The enzyme hydrolysis was carried out at a temperature of between 55-60° C. for 50-60 minutes. The extract was isolated using liquid chromatography to produce a fraction comprising substances with molecular weights <10 kDa, which was then subjected to mass spectrometry analysis to identify peptides present in the fraction. Once the peptides were identified they were analysed using custom bioinformatics software to link the peptides with potential functional properties and/or physiological effects.

The results of the peptide analysis are listed in the table below:

| Antihypertensive peptides | |
|---|---|
| (including ACE inhibitor | Count of Sequence Activity |
| peptides marked as *) | in Sample |
| Phe-Phe | 3 |
| Leu-Asp-Leu | 4 |
| Leu-Glu-Leu | 4 |
| Leu-Gly-Leu | 4 |
| Leu-Leu-Phe* | 18 |
| Leu-Asn-Phe* | 3 |
| Leu-Thr-Phe* | 7 |
| Leu-Trp* | 34 |
| Val-Asp-Phe | 4 |
| Val-Asp-Trp | 2 |
| Val-Glu-Phe | 2 |

| | | | |
|---|---|---|---|
| Glycine | Gly | Proline | Pro |
| Alanine | Ala | Valine | Val |
| Leucine | Leu | Isoleucine | Ile |
| Methionine | Met | Cysteine | Cys |
| Phenylalanine | Phe | Tyrosine | Tyr |
| Tryptophan | Trp | Histidine | His |
| Lysine | Lys | Arginine | Arg |
| Glutamine | Gln | Asparagine | Asn |
| Glutamic Acid | Glu | Aspartic Acid | Asp |
| Serine | Ser | Threonine | Thr |

These results show that the isolated <10 kDa fraction comprised a number of peptides that are likely to contribute to the antihypertensive and/or ACE inhibition activities in the extract. These are small peptides such as dipeptides and tripeptides consisting of two to three amino acids. The molecular weight range of each of these peptides is likely to be between about 200-500 Daltons. There is a higher occurrence of tripeptide Leu-Leu-Phe and dipeptide Leu-Trp in the sample than other peptides. It is envisaged that one or more of the ACE inhibitory peptides identified in the table above, or a combination of these peptides could be further isolated and concentrated, for example, by a further ultrafiltration step to obtain a <1 kDa molecular weight fraction, for use as ACE inhibitory peptides either as extracts on their own or in various compositions for the treatment, regulation or prevention of high blood pressure and/or hypertension.

Further studies have since shown that there are hundreds of cryptides or potential bioactive peptides that may be responsible for the antihypertensive and/or ACE inhibition activities demonstrated by the extract of the invention (see Example 7). It is likely that the type and number of peptides present in the extract of the invention will vary according to the method of manufacture of the extract and/or the starting material (crude extract) that was used to obtain the isolated fraction. For example, if enzyme hydrolysis is used to make the crude extract, the enzymes used in that process, and the time and temperature parameters are likely to have a bearing on the type and number of peptides present in the resulting isolated 10 kDa or smaller fraction. It is evident however that enzyme hydrolysis is not essential for obtaining an extract with antihypertensive peptides and/or ACE inhibition peptides, since slightly lower but still significant numbers of these peptides are present in extracts prepared by other methods.

### Example 3—Antioxidant Activity

Oxidative processes and formation of free radicals are thought to be a cause or contributor to many different types of diseases or health conditions. In addition, oxidation of foods is one of the main causes of food deterioration. In the food and pharmaceutical industries, synthetic antioxidants such as butylated hydroxytoluene and others are used to prevent oxidation and food spoilage. However, use of synthetic antioxidants is strictly regulated due to potential health risks of these compounds. The isolation and use of natural antioxidants is therefore beneficial in terms of providing various health benefits as well as providing natural alternatives to synthetic antioxidants used in food, cosmetics and pharmaceutical products.

In order to investigate the potential antioxidant activity of non-lipid green-lipped mussel extracts, the same nine samples described in Example 1 were also tested for antioxidant activity.

The antioxidant activity of the samples was tested using the DPPH scavenging method (i.e. by using the stable free radical 2,2-Diphenyl-1-(2,4,6-trinitrophenyl)hydrazyl as a substrate). The DPPH solution was prepared in 0.1 mM ethanol and kept in the freezer in the dark before use. The positive control was ascorbic acid prepared as 0.1 mg/ml in a buffer containing citric acid and NaHPO₄ (pH 5). Equal amounts of sample solution and DPPH solution were added together, and the assay tube or plate was incubated for 30 minutes in the dark, followed by absorbance measurement at 517 nm on a spectrophotometer. In the blank control experiment of each sample, DPPH was replaced with ethanol. In the DPPH blank control experiment, the sample was replaced with the media (water or solvent) the sample was prepared in.

The scavenging activity (DPPH inhibition %) is calculated by percentage of the absorbance from the sample versus the DPPH only:

$$DPPH inhibition \% = [ 1 - \frac{( A sample - A sample blank )}{( A DPPH - A DPPH blank )} ] \times 100$$

All samples were tested at a concentration of 10 mg/ml. The results showed that all samples had antioxidant activity (above 80% inhibition in all samples). The results are summarised in the table below:

TABLE 3
IC₅₀ values for DPPH scavenging activity
of each hydrophilic extract sample

| Sample | 1 H | 2 H | 3 H | 4 H | 5 H | 6 H | 7 H | 8 H | 9 H |
|---|---|---|---|---|---|---|---|---|---|
| IC₅₀ | 6.90 | 2.96 | 3.72 | 3.01 | 3.02 | 8.05 | 3.32 | 4.23 | 5.69 |
| (mg/ml) | | | | | | | | | |

Notably, the dried mussel compositions produced by the enzymatic processing method described in PCT/NZ2017/050167 (particularly samples 4, 5 and 7) showed very good antioxidant activity. Compositions produced by this method also provide much higher yields of hydrophilic components (about 70% yield in comparison to about 30% yield in other processes) so a hydrophilic fraction obtained by this processing method will have a higher overall bioactivity.

The corresponding lipid extracts of each of the above hydrophilic extracts was also tested for DPPH inhibition activity, as a comparison, and each lipid extract sample showed a good (but slightly lower) level of activity. Whilst both fractions contribute to the overall DPPH inhibition activity, it was surprisingly found that the hydrophilic extracts exhibited higher activity, and given that higher yields of hydrophilic fractions can be obtained from crude mussel extracts or compositions, than lipid fractions, a more effective biologically active extract comprising the non-lipid or hydrophilic components can be produced from less raw material.

### Example 4—Antioxidant Activity of Fractionated Samples

Sample A of Example 2 was tested for DPPH scavenging activity. Both the retentate after 10 kDa membrane filtration (>10 kDa) and the filtrate after 10 kDa membrane filtration (<10 kDa) at 5 mg/mL were tested, using the same method as described in Example 3. The results of the test are shown in the following table.

| Sample | >10 kDa fraction | <10 kDa fraction |
|---|---|---|
| A | 32.11% | 55.05% |

This test shows that the DPPH scavenging activity was higher in the isolated <10 kDa fraction.

### Example 5—Antioxidant Compounds

In order to further identify which components in the hydrophilic extract samples described in Example 3 were responsible for the DPPH inhibition activity, sample 3 was selected for further investigation. The sample was fractionated by size exclusion chromatography using Superdex 75. A 2.5 ml (50 mg) aliquot of sample 3 was loaded each time and eluted with PBS buffer (50 mM phosphate buffer at pH7.2 with 150 mM NaCl) for 2 column volumes (2×120 ml) at a flow rate of 1 ml/min. Fractions were collected at 5 ml each and the first 36 fractions were analysed for protein and sugar concentrations and DPPH inhibition. SDS-PAGE analysis with silver staining was done for all fractions containing protein and peptides. Fractions with high DPPH scavenging activity were combined from 5 runs (a total of 250 mg load) and concentrated via freeze-drying. Dried fractions were reconstituted with water at 10-fold less than their original volumes. DPPH scavenging activity analysis and protein determination were performed again on these concentrated fractions to confirm their DPPH inhibition activity.

The results of the Superdex 75 fractionation chromatography are shown in FIG. 1. The Superdex 75 fractionation revealed several fractions of test sample 3 which exhibited high DPPH inhibition activity. The protein concentrations, sugar concentrations and DPPH inhibition activity in each fraction are displayed in FIGS. 2 and 3. Most proteins were eluted in the first 30-80 ml (half-column volume), and a small amount of proteins were collected at the end of one-column volume between 105-120 ml. The sugar contents were eluted mostly in two regions: A8-A9 and B8-B11. It was found that the DPPH scavenging activity did not follow the protein concentration trace, but aligned with the sugar trace to some extent in the fractions. The DPPH scavenging activity peaked at fraction B10, with two adjacent fractions B9 and B11 also demonstrating good activity. The fractions displaying the highest DPPH inhibition activity were concentrated via freeze drying and then reconstituted with water to yield a 10-fold by volume concentrated sample. DPPH inhibition was tested again and the concentrates of B8, B9, B10, B11 and B12 demonstrated 2-4 fold improvement in activity. This confirms the DPPH scavenging activity in these fractions.

All the protein-containing fractions were analysed by SDS-PAGE with silver-staining for visualisation and the results are shown in FIGS. 4 and 5. FIG. 4 shows that test sample 3 has major protein bands of less than 75 kDa which were eluted in fractions between A8 and B2. The fractions A12, B1 and B2 showed major protein bands around 28-38 kDa. Smaller peptides of less than 28 kDa, particularly around 6 kDa were eluted in fraction B8, but not so clearly in fractions B9, B10, B11 and B12 (see FIG. 5). The low quantities of peptide in B9, B10, B11, and B12 is most likely to be due to the presence of much smaller peptides and/or free form amino acids and/or other non-protein molecules.

If the DPPH scavenging activities were normalised by the total concentration of protein and sugar in each fraction, B11 would possess the highest activity (see FIG. 6). The protein contents in B11 and B12 are negligible (lower than the protein detection limit), while sugar contents are at 39 μg/ml and 53 μg/ml (refer Table 4 below).

TABLE 4
Data of DPPH activities versus the sum of sugar and
protein quantity and sugar and protein
concentrations in some Superdex 75 fractions.

| Fractions | sugar | protein | | |
|---|---|---|---|---|
| Concentrates | (mg/ml) | (mg/ml) | DPPH % | ~DPPH %/mg/ml |
| B8C | 1.315 | 0.482 | 73.2% | 40.8% |
| B9C | 4.473 | 0.894 | 88.2% | 16.5% |
| B10C | 1.148 | 0.321 | 79.7% | 54.3% |
| B11C | 0.390 | 0.023 | 58.2% | 141.9% |
| B12C | 0.532 | 0.092 | 32.7% | 52.6% |
| B8C-B12C | 1.572 | 0.362 | 66.4% | 61.2% |
| combined | | | | |
| (12.5 ml) | | | | |

~calculated value based on total concentration of protein and sugar.

Further analysis was conducted on four of the concentrated active fractions obtained from Superdex 75 separation, that is, B9C, B10C, B11C and B12C. These fractions were <10 kDa molecular weight fractions (see FIG. 5) and were analysed by HPLC-MS with a C18 column. LC-MS analysis was conducted on a Waters Alliance 2795 UPLC coupled with a diode array detector and a QToF Premier Tandem mass spectrometer (MS). The C18 column was Kinetex XB-C18 (100×3.0 mm, 2.6μ). B10C and B11C were selected for further fractionation by a C18 preparative column (Prodigy 5 u, ODS(3) 100 A, 10×250 mm), based on their higher DPPH activity after normalisation by protein and sugar quantities. C18 fractionation was performed on a Gilson preparative HPLC system with a UV/VIS detector (Gilson 156) and a fraction collector (GX-241). The chromatograms were monitored by three wavelengths (210 nm, 280 nm and 360 nm). The fractions were collected for every 5 ml. Selected fractions after C18 fractionation were analysed by LC-MS as described above. The chromatograms of B10C and B11C fractionation on C18 are shown in FIGS. 7 and 8. The chromatograms showed that B10C has 4 major peaks after C18 separation, F5, 6, 7 and F20.

Like B10C, the fractionation of B11C was also centred in two regions: 3 earlier peaks around 5 min retention time point and 5 peaks around 10 min retention time point. Interestingly, UV traces demonstrated different intensities for each peak at similar retention time as in B10C, indicating the likelihood of different compounds. LC-MS results for the Superdex 75 fractions, namely B9C, B10C, B11C and B12C are displayed in FIGS. 9 to 12. All LC-MS data for B9C, B10C, B11C and B12C suggests that there are several biologically active compounds in each of these fractions.

The UV and MS charts in FIG. 9 show that fraction B9C contains sugars. These are likely to be tetrasaccharides or a mixture of oligosaccharides up to four 6-carbon sugars. A few amino acids are also shown in the late peaks, which may be present in free-form or as glycopeptides.

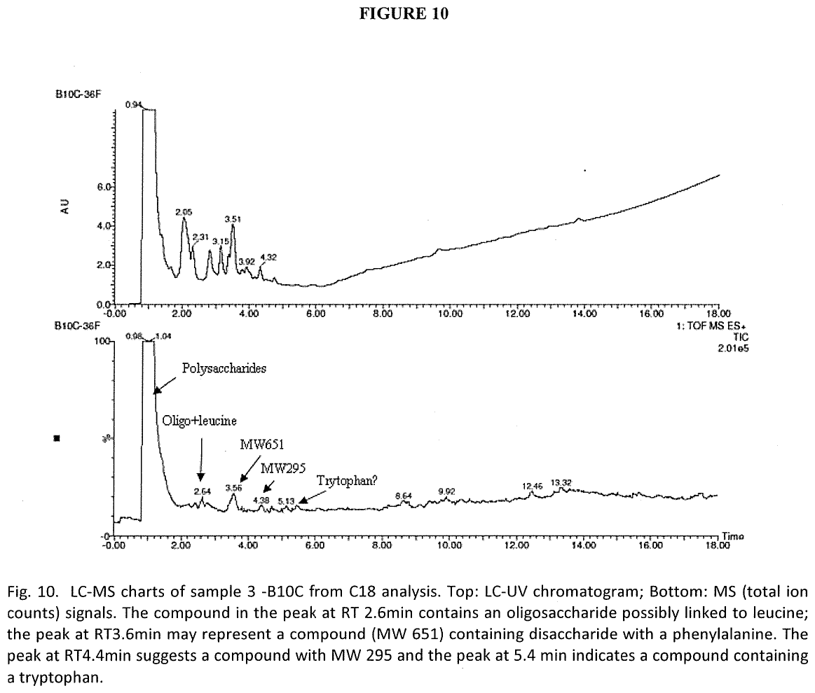

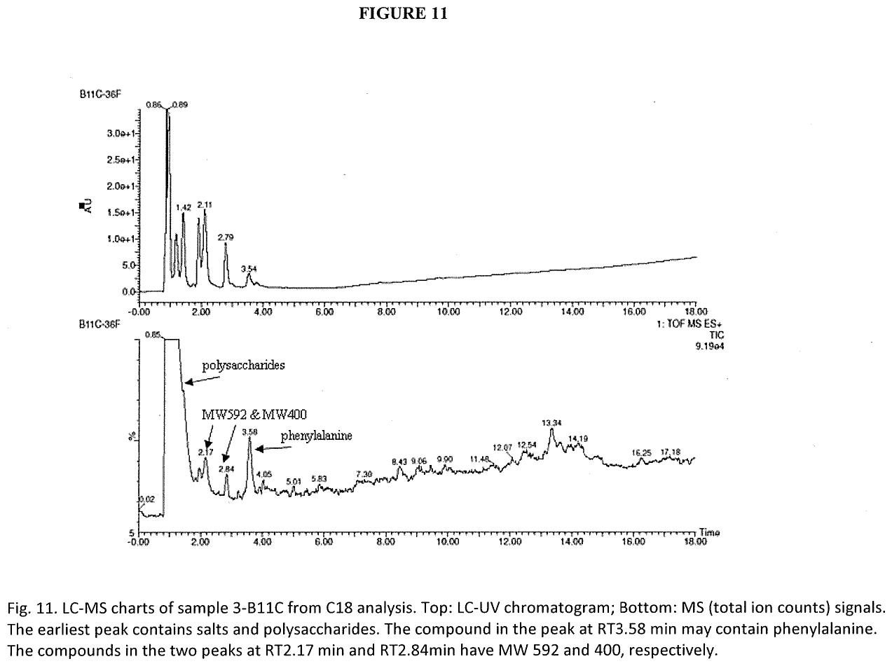

The chromatograms of B10C and B11C show some level of similarity in the retention times of the major peaks (FIGS. 10 and 11). However, further analysis of the MS data demonstrated that the two samples have different compositions. The peak at RT 2.6 min in B10C may contain a sugar compound that is likely to have two 6-C sugar units and one 5-C sugar unit. The presence of a leucine mass fragment suggests either it exists as free form or is attached to the sugar containing compound. The peak at RT3.6 min may also be a sugar compound that is likely to have two 6-C sugar units. The mass fragments of two amino acids, phenylalanine and tryptophan, were present, which could either be small glycopeptides or free form amino acids.

In FIG. 11, B11C shows peaks at RT2.17 min and RT2.84 min, respectively. It is putated that these peaks relate to compounds with molecular weights (MWs) of 592 and 400 respectively. Due to all the MS fragments showing odd numbers, it is certain that these two peaks contain even numbers of nitrogen (e.g. 0, 2, 4 or 6). The later peak at RT3.58 min may contain phenylalanine.

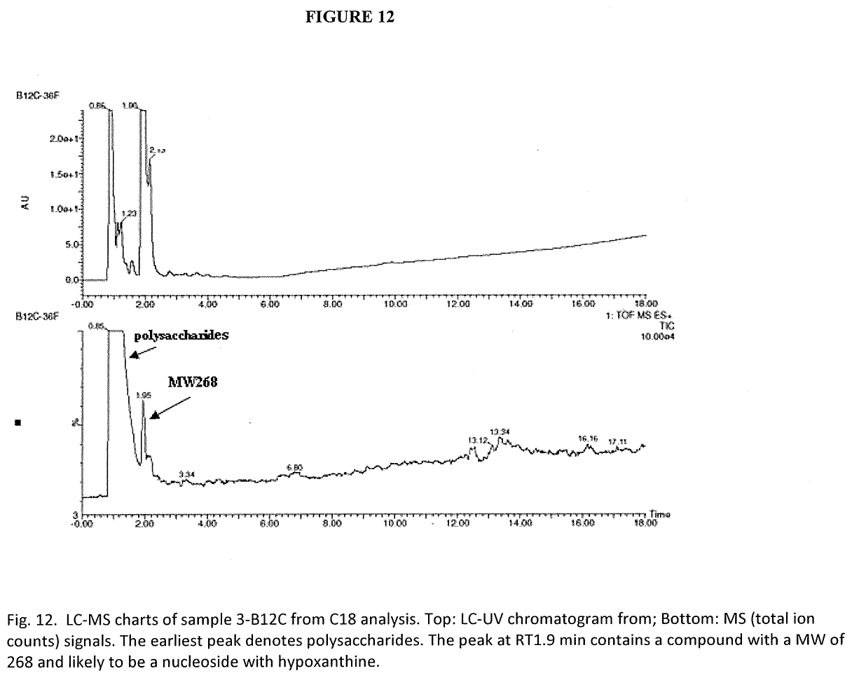

Both UV and MS chromatograms of B12C (FIG. 12) show only two major peaks. The earlier peak is confirmed as small sugars as found in B10C and B11C; the main compound in the second peak at RT1.95 min has a molecular weight of 268 possibly formed by a 5-C sugar and a purine derivative-hypoxanthine.

Further fractionation of B10C and B11C was carried out on a preparative C18 column. The following fractions were analysed further by LC-MS via a C18 analytical column: B10C-F7, B11C-22, 23 and 24. The LC-MS data of fraction B10C-F7 showed an oligosaccharide containing compound which is possibly linked to a histidine and has a MW of 663 (FIG. 13). This compound is dominant in F7-10 fold larger in quantity than others, evaluated by its UV and MS signal strength. The MS data of the other three peaks in F7 suggests that the first one (RT1.9 min) is a compound of MW244, the second one (RT2.7 min) contains a molecule of MW145, and the last one (RT 3.9 min) matches the MS fragmentation of a free-form histidine.

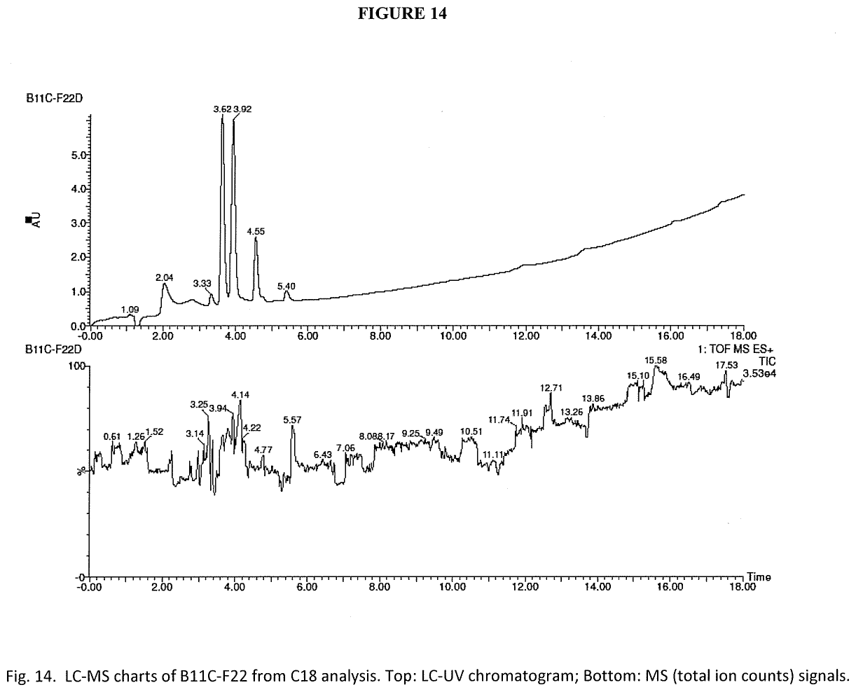

The chromatograms of the active fractions from B11C fractionation, F22-24, showed less or depleted polysaccharides peaks (RT<2 min). F22 and 23 showed similar chromatograms with four major peaks in the UV trace after C18 chromatography, eluted between 3.5 min and 5.6 min (FIGS. 14-16), however, with different peak height ratios between them. The first two peaks in F22 are higher than the latter two peaks, while in F23 there is a balanced distribution between the 4 peaks. F24 only has one major UV peak at RT4.5 min.

The MS analysis of the first peak (RT3.7 min) in both F22 and F23 suggests a compound of MW 290 (MW 268 without Na) containing a 5-C sugar and a possible nucleoside—a nucleobase like compound linked with 5C-sugar. This compound was also found in the B12C analysis. The second peak (RT4.1 min) may be a molecule containing at least one 6C sugar unit and with a total MW of 592, and possibly contain even numbers of nitrogen. The third peak (RT4.7 min) may be a molecule with a total MW of 400 containing a unit of mass 136 (hypoxanthine) and a fragment MS at 127 (thymine). The last peak (RT 5.4 min) contains phenylalanine (MW 165) in its free-form.

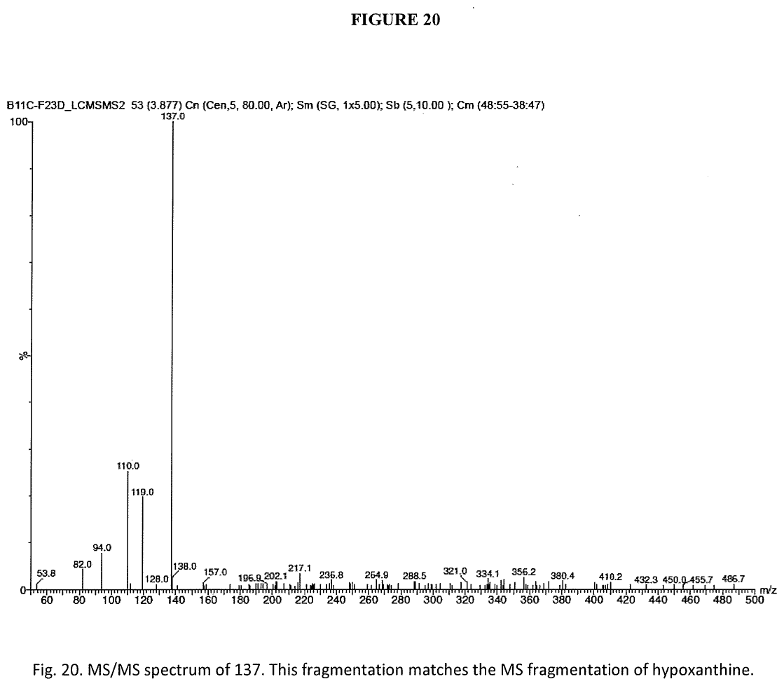

To further verify these four molecules (MWs 268, 592, 400, and 165), MS/MS analysis of these four compounds targeting their MWs were conducted in positive mode (MS+). The results (FIGS. 17 to 19) revealed that the first two compounds both contain a main fragment of MS137-likely to be hypoxanthine (containing 2 nitrogen), suggesting that they may be structure-related. Indeed, there is a 6C-sugar unit difference between MS269 and MS431, suggesting that the first compound is two 6C-sugar units less than the second compound. Further MS/MS analysis of MS137 confirms that it is hypoxanthine (FIG. 20).

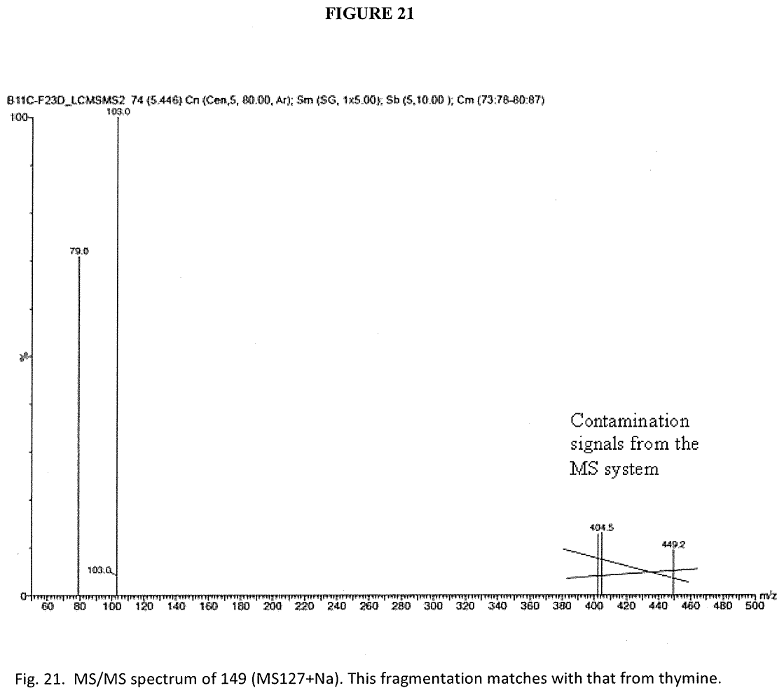

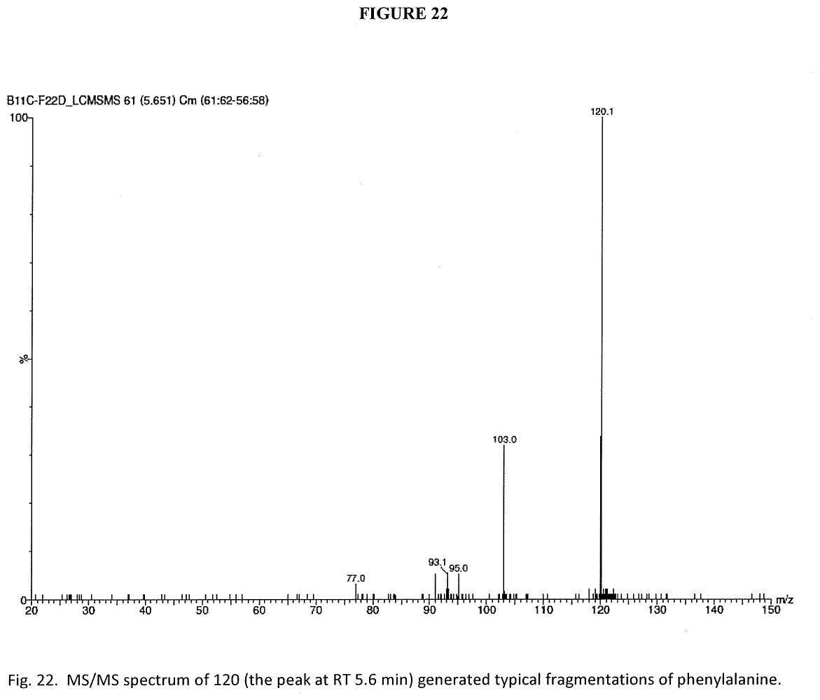

In the MS/MS of 269, the mass difference between MS269-137 is a loss of 5C-sugar (132), suggesting that the first peak in B11C-22 may represent a nucleoside and the second compound is a nucleoside with additional two 6-C sugar. MS/MS spectrum of the third peak has two main fragments of MS265 and MS149, a loss of mass 116—a deoxyl 5-carbon sugar (deoxyribose), further MS/MS of 149 (127+Na+) generated a 103 and 79, matching the MS+ fragmentation of thymine (FIG. 21). These MS data suggest that the third peak may be a thymidine (MW242) with a hypoxanthine in sodium salt form (total MW 400). The MS/MS of the fourth peak demonstrated a typical mass fragmentation of a phenylalanine in its free-form (MW 165) (FIG. 22).

For the structure analysis of the three unknown compounds in F22-24, the UV spectrums were scanned, respectively. The first two peaks (RT 3.5 min and 4.0 min) displayed the same optimum UV absorbance at 248 nm, further supporting the structural similarity between these two compounds. The third peak has an optimum UV absorbance at 267 nm which indicated a ketone containing compound as in thymidine. From Sigma Aldrich (www.sigmaaldrich.com), the maximum UV absorbance for hypoxanthine is 249 nm, and for thymidine is 267 nm, supporting the conclusions obtained from the LC-MS/MS analysis on the three unknown compounds in B11C.

The UV spectrums were also scanned for the four peaks in B 10C-F7. The dominant peak (RT 1.3 min) has an optimum UV absorbance at 273 nm, the second peak (RT 1.8 min) at 318 nm, the third peak (RT2.4 min) at 190 nm which indicates no aromatic ring, and the last peak (RT3.8 min) at 253 nm. This confirms that the compounds in B10C-F7 are different from those in B11C.

These results show that there are a plurality of biologically active compounds likely to be responsible for the antioxidant activity of the extract of the invention. It appears that the lead bioactive compounds in respect of antioxidant activity have molecular weights of less than 1 kDa (for example, the compounds with MW 663, MW 165, MW 268, MW 592 and MW 400). Accordingly a concentrated extract of the invention comprising a molecular weight fraction of <1 kDa is likely to have significant antioxidant properties. The antioxidant activity is most likely to be provided by a combination of free form amino acids such as histidine and phenylalanine, some small peptides or cryptides, sugars and sugar-containing compounds such as nucleosides and their derivatives, and nitrogen-containing compounds such as the purine derivative hypoxanthine.

### Example 6—Free Amino Acid Analysis of <10 kDa Fractions

### Methodology

Ten fractionated mussel powder samples as prepared below were analysed for free amino acid content.

| Sample No's | Description of Samples |
|---|---|
| 3R; 5R; 9R | Extract produced by mechanical opening of mussels, |
| | removal of meat, homogenising of meat and spray or |
| | freeze drying. Dried extract was rehydrated in water |
| | and fractionated by centrifugation to recover the |
| | hydrophilic fraction, which was then subjected to |
| | membrane filtration using 10kDa ultracentrifugation. |
| 4R; 6R; 16R; | Extract produced using enzymatic process described in |
| 18R; 24R; | PCT/NZ2017/050167 to produce a liquid whole mussel |
| 25R | composition, followed by spray or freeze drying. |
| | Dried extract was rehydrated in water and fractionated |
| | by centrifugation to recover the hydrophilic fraction, |
| | which was then subjected to membrane filtration |
| | using 10kDa ultracentrifugation. |
| 26R | Extract produced by mechanical opening of mussels, |
| | removal of meat, homogenising of meat and spray |
| | or freeze drying. Lipids were removed by CO2 |
| | supercritical extraction. Remaining hydrophilic |
| | fraction was subjected to membrane filtration |
| | using 10kDa ultracentrifugation. |

20 mg of each sample were first accurately weighed out in pyrolyzed glass vials. 300 uL of 0.1 M HCl was then added to each sample, vortexed briefly and then and sonicated for 3 hours. After the sonication step, the samples were centrifuged at 40,000 g for 1 hour. The supernatants were filtered through a 0.45-micron filter (Advantec) and the filtrate collected in fresh Eppendorf tubes, dried under vacuum and then dissolved in 1 mL Na2HPO4 buffer, pH 7.4. Derivatisation of the free amino acids was carried out with AccQ-Tag reagent (Waters).

Free Amino analysis was performed using an Ultimate 3000 HPLC system (Dionex). 5 uL of the derivatised samples were injected onto a Thermofisher XL C18 Accucore Column (4.6 mm i.d×250 mm, 4 um particles), protected with as C18 guard column. The separation of the amino acids was performed at 37° C. using a flow rate of 1.0 mL/min using a gradient from 1.5-16.5% mobile phase B over 52 min. Mobile phase A was AccQ-Tag eluent A in LC grade water, and mobile phase B was 100% LC grade Acetonitrile. An excitation wavelength of 250 nm and emission wavelength of 395 nm were used for the quantitative analysis using a fluorescence detector (Dionex ultimate FLD 3000).

### Results

Results from the amino acid analysis are shown in FIG. 24 as absolute quantities (mg/100 mg). The results show that all of the samples tested had a similar range and quantity of amino acids. The w/w percentage of total free form amino acids in each sample ranged from about 1-10%, the majority of samples having between 7-9% w/w of free form amino acids.

The most prominent amino acid present in each sample was Arginine, followed by Glycine. The other amino acids were present in much lower quantities. The percentage of Arginine in each sample was between about 30-50% relative to the other amino acids. The percentage of Glycine in each sample was between about 15-35% relative to the other amino acids.

Arginine is an essential amino acid. Studies have shown that Arginine reduces blood pressure. A meta-analysis showed that L-arginine reduces blood pressure with pooled estimates of 5.4 mmHg for systolic blood pressure and 2.7 mmHg for diastolic blood pressure. (See Dong J Y, Qin L Q, Zhang Z, Zhao Y, Wang J, Arigoni F, Zhang W (December 2011). “Effect of oral L-arginine supplementation on blood pressure: a meta-analysis of randomized, double-blind, placebo-controlled trials”, review, American Heart Journal, 162 (6): 959-965.) It has also been shown that Supplementation with L-arginine reduces diastolic blood pressure and lengthens pregnancy for women with gestational hypertension, including women with high blood pressure as part of pre-eclampsia_(Gui S, Jia. J, Niu X, Bai Y. Zou H, Deng J, Zhou R (March 2014). “Arginine supplementation for improving maternal and neonatal outcomes in hypertensive disorder of pregnancy: a systematic review”. (review). Journal of the Renin-Angiotensin-Aldosterone System, 15 (1): 88-96).

Accordingly, since the <10 kDa fractions each comprise a significant amount of Arginine in free form, this could play a key role in the antihypertensive activity of the extracts of the invention.

The second most prominent amino acid in each sample was Glycine which is responsible for many different muscle, cognitive and metabolic functions in the body.

The samples also comprised other essential amino acids, including Histidine, Isoleucine, Leucine, Lysine, Methionine, Phenylalanine, Threonine, and Valine. The presence of free form essential amino acids in the extracts of the invention is advantageous as free form amino acids are easier to absorb by the body. Accordingly, extracts of the invention could be used in dietary and nutritional supplements and as functional food and beverage products to provide a source of essential amino acids.

### Example 7—Peptide/Cryptide Analysis of <10 kDa Fractions

Cryptides are bioactive peptides hidden or encrypted within proteins that exert a positive physiological function following consumption. Cryptides generally contain between 3 and 20 amino acid residues, and their bioactivities are based on the inherent amino acid composition and location within the peptide sequence. They are inactive in the sequences of their parent proteins but may be released by a number of methods including enzymatic hydrolysis, fermentation or other food processing methods, or by gastrointestinal (GI) digestion. To exert a positive physiological function, cryptides must cross the intestinal barrier and survive enzyme degradation in the GI tract following consumption. Cryptides are often multifunctional and can exert several beneficial physiological effects at different target sites when liberated in the human body, depending on their amino acid sequence.

### Methodology

Six fractionated mussel powder samples as prepared below were analysed for cryptide hence potential bioactive peptide content.

| Sample No's | Description of Samples |
|---|---|
| 5R; 9R | Extract produced by mechanical opening of mussels, |
| | removal of meat, homogenising of meat and spray or |
| | freeze drying. Dried extract was rehydrated in water and |
| | fractionated by centrifugation to recover the hydrophilic |
| | fraction, which was then subjected to membrane |
| | filtration using 10kDa ultracentrifugation. |
| 4R; 6R; 18R; | Extract produced using enzymatic process described in |
| 27R | PCT/NZ2017/050167 to produce a liquid whole mussel |
| | composition, followed by spray or freeze drying. |
| | Dried extract was rehydrated in water and fractionated |
| | by centrifugation to recover the hydrophilic fraction, |
| | which was then subjected to membrane filtration |
| | using 10kDa ultracentrifugation. |

### Sample Preparation

Samples were fully dissolved in LC MS water by sonication for 5 min to produce a 20 mg/ml solution. The solutions were then centrifuged at 10,000 g for an hour at 4° C. The clear supernatant obtained was then diluted four times with 5% (v/v) acetonitrile and 400 ul of each of the diluted solution was filtered through 10K MWCO filters (Pall) by centrifugation for 30 minutes at 4° C. at 10,000 g. The filtrate was then dried in a vacuum concentrator and resuspended in 50 ul 0.1% formic acid for mass spectrometry analysis.

### LC-MS and LC-MS/MS Analysis

LC-MS was performed on a nanoflow Ultimate 3000 UPLC (Dionex) coupled to Impact II mass spectrometer equipped with a CaptiveSpray source (Bruker Daltonik, Bremen, Germany). For each sample, 1 μL of the sample was loaded on a C18 PepMap100 nano-Trap column (300 μm ID×5 mm, 5 micron 100 Å) at a flow rate of 3000 nl/min. The trap column was then switched in line with the analytical column ProntoSIL C18AQ (100 μm ID×150 mm 3-micron 200 Å). The reverse phase elution gradient was from 2% to 20% to 45% B over 60 min, total 88 min at a flow rate of 600 nL/min. Solvent A was LCMS-grade water with 0.1% formic acid; solvent B was LCMS-grade ACN with 0.1% formic acid. The samples were measured in data-dependent MS/MS mode, where the acquisition speed was 2 Hz in MS and 1-5 Hz in MS/MS mode depending on precursor intensity. The analysis was performed in positive ionization mode with a dynamic exclusion of 60 sec.

### Peptide Identification

Peptides were identified using PeaksX studio (Bioinformatics solutions Inc.). An error of 10.0 ppm for the precursor mass and 0.2 Da for the fragment ion was allowed. The compounds were searched initially via de novo followed by a search against the NCBI Mytilus database. The search parameters included no enzyme and variable modifications were oxidation of methionine and deamidation of asparagine or glutamine. The compounds were then searched further for other modifications and again with Spider for single point amino acid substitutions.

### Bioactive Peptide Search

Custom Visual Basic for Applications (VBA) macros were used to search for putative bioactive matches of peptides from 69,326 peptide entries compiled from various databases including BIOPEP, PeptideDB, APD2 and EROP, with additional sequences obtained from relevant scientific literature.

### Results

The analysis revealed exact matches for the following peptide sequences in at least two of the samples tested: Leu-Val-Ser-Lys and Leu-Tyr-Glu-Gly-Tyr. These peptide sequences were identified as antioxidant peptides in the Bioinformatics analysis.

The total number of cryptides or potential bioactive peptides identified in each sample is shown in FIG. 25. The results show that hundreds of potential bioactive peptides were identified in each sample (at least 600). Notably, higher numbers of cryptides were identified in the samples prepared using enzyme hydrolysis, suggesting that extracts of the invention obtained from a crude extract prepared using enzyme hydrolysis will have higher numbers of bioactive peptides and a potentially wider range of bioactivities.

The potential bioactivities of the cryptides was analysed using Bioinformatics as described above, and those cryptides that could be linked to certain bioactivities are represented in FIG. 26, which shows the types and proportions of potential bioactive peptides present in each sample.

The results show that each sample comprised high numbers of cryptides which could be attributed to a range of bioactivities including antihypertensive/ACE inhibition activity; antioxidant activity; antimicrobial activity; antiviral activity and antiparasitic activity. The analysis revealed that a high proportion of potentially bioactive antihypertensive and/or ACE inhibition peptides are present in the extract of the invention.

The isolated <10 kDa fractions contained between about 34-38% potentially bioactive antihypertensive and/or ACE inhibitor peptides.

The analysis also shows that the isolated <10 kDa fractions contained between about 5-8% potentially bioactive antioxidative peptides, and between about 4-8% potentially bioactive antimicrobial peptides. Smaller amounts of potentially bioactive antiviral and antiparasitic peptides were also identified in each sample. It is therefore envisaged that the extracts of the invention could be used either alone or in various compositions for providing these bioactive properties.

### Advantages

a) The bioactive extracts of the invention demonstrate a good level of biological activity, and therefore can be utilised in many ways to make high value products (including various products with multiple health benefits) instead of being discarded or used in low value by-products;
b) Much higher yields of non-lipid or hydrophilic components can be obtained from green-lipped mussels (compared with their lipid extract counterparts) therefore isolated extracts and compositions with higher efficacy can be made using less raw material;
c) Compositions comprising non-lipid components have an improved smell and taste (less fishy flavour) since the lipid fraction comprising fat soluble aroma or volatiles responsible for these sensory attributes has been removed;
d) The extracts of the invention are suitable for use in many applications and in many different product formats. They can potentially be used in various pharmaceutical, veterinary, nutraceutical (such as dietary supplements), cosmetic, or food applications, for example to increase the nutritional value of food products and also in the development of functional foods;
e) The extracts of the invention may provide a suitable natural alternative to synthetic drugs or compounds (such as synthetic ACE inhibitors and synthetic antioxidants) which often have undesirable side effects or health risks. They are also less expensive to produce.

### Variations

Throughout the description of this specification, the word “comprise” and variations of that word such as “comprising” and “comprises”, are not intended to exclude other additives, components, integers or steps.

It will of course be realised that while the foregoing has been given by way of illustrative example of this invention, all such and other modifications and variations thereto as would be apparent to persons skilled in the art are deemed to fall within the broad scope and ambit of this invention as is hereinbefore described.

1.-24. (canceled)

25. A biologically active non-lipid extract obtained from New Zealand green-lipped mussels (Perna canaliculus), wherein the extract exhibits biological activity selected from one or more of antioxidant activity, antihypertensive activity, antimicrobial activity, antiviral activity, and antiparasitic activity.

26. The extract as claimed in claim 25 wherein the extract comprises a plurality of biologically active substances with molecular weights of <100 kDa selected from the group comprising free amino acids; peptides; cryptides; sugars and/or sugar-containing compounds including nucleosides and their derivatives; carbohydrates including glycoconjugates such as glycosides, glycosylamines, glycoproteins, glycopeptides, peptidoglycans; nitrogen-containing compounds including purines; phenolic compounds; minerals; metabolites including small molecule metabolites.

27. The extract as claimed in claim 25 wherein the extract consists of an isolated molecular weight fraction of <10 kDa or <1 kDa.

28. The extract as claimed in claim 27, wherein the extract comprises between about 1-10% by weight of free form amino acids.

29. The extract as claimed in claim 28 wherein the extract comprises a relatively higher proportion of amino acids Arginine and/or Glycine.

30. The composition comprising an extract as claimed in claim 25.

31. The composition as claimed in claim 30 wherein the composition is a pharmaceutical, nutraceutical, veterinary, cosmetic, cosmeceutical, food composition, functional food or beverage, functional food or beverage ingredient, functional food or beverage additive, or a dietary supplement.

32. The composition as claimed in claim 30 wherein the composition is an antihypertensive composition.

33. The composition as claimed in claim 32 wherein the antihypertensive effect is provided by one or more free amino acids and/or one or more cryptides and/or one or more peptides present in the extract.

34. The composition as claimed in claim 33 wherein the extract comprises at least 30% potentially bioactive antihypertensive peptides.

35. The composition as claimed in claim 33 wherein the extract comprises a plurality of peptides, at least one of which is selected from the group comprising peptides having amino acid sequences: Phe-Phe; Leu-Asp-Leu; Leu-Glu-Leu; Leu-Gly-Leu; Leu-Asn-Phe; Leu-Thr-Phe; Leu-Trp; Val-Asp-Phe; Val-Asp-Trp; Val-Glu-Phe; Leu-Leu-Phe; Leu-Trp-Phe.

36. The composition as claimed in claim 30 wherein the composition is an antioxidant composition.

37. The composition as claimed in claim 36 wherein the extract comprises one or more peptides selected from peptides having amino acid sequences: Leu-Val-Ser-Lys and/or Leu-Tyr-Glu-Gly-Tyr.

38. The composition as claimed in claim 37 wherein the extract comprises at least 5% potentially bioactive antioxidative peptides.

39. A method of manufacturing a composition for the treatment, regulation or prevention of high blood pressure or hypertension, comprising introducing an effective amount of a therapeutically amount of a non-lipid extract as claimed in claim 25.

40. The method of treating, regulating or preventing high blood pressure or hypertension by administering to a subject in need thereof, a therapeutically effective amount of a composition as claimed in claim 30.

41. An ACE inhibitory peptide isolated from New Zealand green-lipped mussels (Perna canaliculus), wherein the peptide comprises an amino acid sequence selected from the group consisting of Leu-Leu-Phe; Leu-Asn-Phe; Leu-Thr-Phe; and Leu-Trp.

42. The antihypertensive composition comprising one or more of the peptides as claimed in claim 41.

43. The composition as claimed in claim 30 wherein the composition is an antimicrobial, antiviral or antiparasitic composition.
